(19) United States
(12) Patent Application Publication
(10) Pub. No.: US 2015/0241417 A1
(43) Pub. Date: Aug. 27, 2015

(54) ASSAY DEVICES COMPRISING A POLY(ACID) MEMBRANE, AND METHODS USING THE SAME

(71) Applicant: Clontech Laboratories, Inc., Mountain View, CA (US)

(72) Inventors: George G. Jokhadze, Mountain View, CA (US); Sayantan Mitra, Mountain View, CA (US)

(21) Appl. No.: 14/582,021

(22) Filed: Dec. 23, 2014

Related U.S. Application Data
(60) Provisional application No. 61/943,217, filed on Feb. 21, 2014.

(51) Int. Cl. G01N 33/545

(52) U.S. Cl. CPC G01N 33/545

(57) ABSTRACT

Assay devices that include a poly(acid) membrane are provided. Aspects of the devices include a solid support and a poly(acid) membrane on a surface of the support, where the poly(acid) membrane includes an affinity element. In using the assay devices, a sample is contacted with the poly(acid) membrane and then a signal is obtained from the membrane. Also provided are kits that find use in practicing the methods described herein. The compositions and methods described herein find use in a variety of different applications, including analyte detection applications.

### CROSS REFERENCE To RELATED APPLICATIONS

Pursuant to 35 U.S.C. §119 (e), this application claims priority to the filing date of the U.S. Provisional Patent Application Ser. No. 61/943,217, filed Feb. 21, 2014; the disclosure of which application is herein incorporated by reference.

### INTRODUCTION

Analyte detection in physiological fluids, e.g., blood or blood derived products, is of ever increasing importance to today's society. Analyte detection assays find use in a variety of applications, including laboratory testing (both research and clinical), home testing, etc., where the results of such testing play a prominent role in diagnosis and management in a variety of disease or other conditions.

In addition to physiological fluids, analyte detection in environmental samples, e.g., water, air, etc., are of equal importance. Environmental analyte detection assays find use in a variety of applications, including toxin detection, e.g., in the food industry, the environmental monitoring industry, criminal justice, etc., where the results of such testing play a prominent role in food safety, environmental protection, public safety, and a variety of other functions.

Regardless of the field of use, barriers to effective analyte detection are commonly the detection threshold and the detection rate. Current methods of analyte detection are often hampered by high detection thresholds, requiring relatively large amounts of analyte to allow for signal production, or slow detection rates, requiring relatively long periods of time to allow for signal production indicating some minimal amount of an analyte of interest.

### SUMMARY

Assay devices that include a poly(acid) membrane are provided. Aspects of the devices include a solid support and a poly(acid) membrane on a surface of the support, where the poly(acid) membrane includes an affinity element. In using the assay devices, a sample is contacted with the poly(acid) membrane and then a signal is obtained from the membrane. Also provided are kits that find use in practicing the methods described herein. The compositions and methods described herein find use in a variety of different applications, including analyte detection applications.

### BRIEF DESCRIPTION OF THE FIGURES

FIGS. 1A and 1B show a depiction of an assay device configured as a dipstick according to an embodiment of the invention.

FIGS. 2A and 2B show a depiction of an assay device configured as a disk according to an embodiment of the invention.

FIG. 3 shows a depiction of an assay device configured as a test strip according to an embodiment of the invention.

FIG. 4 shows a depiction of an assay device configured for use in testing a bioreactor or fermenter according to an embodiment of the invention.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The phrases “metal ion affinity composition” and “metal ion affinity complex” are used interchangeably herein and refer to a composition of matter having a polymer or plurality of polymers, e.g., a layer of polymers or a polymeric matrix, bonded to ligand/metal ion complexes. Metal ion affinity compositions of the present disclosure may vary and in some cases make use of a chelating agent, e.g., a ligand, that immobilizes a metal ion to from a ligand/metal ion complex. Chelating agents of the present disclosure may vary and include those agents capable of acting as multidentate ligands, e.g., polydentate chelating ligands, didentate chelating ligands, tridentate chelating ligands, tetradentate chelating ligands, pentadentate chelating ligands, hetaxdentate chelating ligands, etc.

The phrase “chelating ligand” is used herein interchangeably with the term “ligand”. In some instances, the term ligand is used to refer to the individual interactions, i.e. individual bonds, between a multidentate ligand and the central atom to which it binds. For example, a tridentate chelating ligand may be referred to as having three ligands or forming a structure having three ligands with a central atom, e.g., a metal ion. Such ligand bonds are reversible and thus such ligand/central atom complexes may be associated and disassociated, e.g., by changing the environmental conditions within which the chelating ligand and the central atom are present. Central atoms of such complexes may be metal ions (described in greater detail below) and may thus form ligand/metal ion complexes. In certain instances, such ligand/metal ion complexes have affinity for particular analytes, e.g., protein analytes, e.g., particular protein motifs or particular peptides, e.g., a metal ion affinity peptide.

The compositions may be charged or uncharged. A composition is charged when the ligands thereof are complexed with metal ions. Conversely, a complex is uncharged when the ligands thereof are uncomplexed or free of metal ions, but are capable of being complexed with metal ions.

The phrase “metal ion source” refers to a composition of matter, such as a fluid composition, that includes metal ions. As used herein, the term “metal ion” refers to any metal ion for which an affinity agent, e.g., an affinity peptide, has affinity and that can be used for immobilization or detection the affinity agent directly or the detection of a heterologous moiety bound to the affinity agent, e.g., a fusion protein. Such metal ions include, but are not limited to, Ni²⁺, Co²⁺, Fe³⁺, Al³⁺, Zn²⁺ and Cu²⁺. As used herein, the term “hard metal ion” refers to a metal ion that shows a binding preference for oxygen. Hard metal ions include Fe³⁺, Ca²⁺, and Al³⁺. As used herein, the term “soft metal ion” refers to a metal ion that shows a binding preference of sulfur. Soft metal ions include Cu⁺, Hg²⁺, and Ag⁺. As used herein, the term “intermediate metal ion” refers to a metal ion that coordinates nitrogen, oxygen, and sulfur. Intermediate metal ions include Cu²⁺, Ni²⁺, Zn²⁺, and Co²⁺.

As used herein, the term “contacting” means to bring or put together. As such, a first item is contacted with a second item when the two items are brought or put together, e.g., by touching them to each other.

The term “sample” as used herein refers to a fluid composition, where in certain embodiments the fluid composition is an aqueous composition. Also encompassed are those fluid samples generated by contacting a solid, e.g., a surface, a powder, etc., or gas, e.g., air, with a fluid, e.g., by dissolving a solid or gas in a fluid. As used herein, a sample may be a research experiment sample, e.g., a sample generated in a research laboratory, or an environmental sample, e.g., a sample acquired from the natural environment or a domestic, agricultural, or industrial environment.

As used herein, the phrase “in the presence of” means that an event occurs when an item is present. For example, if two components are mixed in the presence of a third component, all three components are mixed together.

The phrase “oxidation state” is used in its conventional sense, see e.g., Pauling, General Chemistry (Dover Publications, NY.) (1988).

The terms “affinity peptide,” “high affinity peptide,” and “metal ion affinity peptide” are used interchangeably herein to refer to peptides that bind to a metal ion, such as a histidine-rich or HAT peptides. The term “affinity tagged polypeptide” refers to any polypeptide, including proteins, to which an affinity peptide is fused, e.g., for the purpose of immobilization or detection.

The terms “heteropolymer” and “copolymer” are used interchangeably herein to refer to those polymers derived from at least two species of constituent units, i.e. monomers, and may be defined as to how the different species of constituent units are arranged. For example, copolymers may be alternating copolymers wherein each unit of the copolymer alternates with one or more different units (e.g., -X-Y-(X-Y-)ₙ . . . , -X-Y-Z-(X-Y-Z-)ₙ . . . , etc.). Alternatively, copolymers may be periodic copolymers wherein units of the copolymer are arranged in repeating sequence (e.g., -X-X-Y-(X-X-Y-)ₙ . . . , -X-Y-Z-Z-Y-(X-Y-Z-Z-Y-)ₙ . . . , -(X-Y-X-Y-Y-X-X-X-X-Y-Y-Y-)ₙ . . . , etc.). Periodic copolymers may be block copolymers wherein the constituent units within a species tend to be bound to another member of the same species (e.g., -(X-X-X-X-X-X-)ₙ-(Y-Y-Y-Y-Y-Y-Y-)ₙ . . . ). Copolymers may be statistical copolymers in which the sequence of constituent units follows a statistical rule, e.g., random copolymer (e.g., copolymer where any position along the copolymer chain has an equal probability of being occupied by monomer X or monomer Y proportional to the relative amounts of monomer X and Y in the whole polymer), gradient copolymer (e.g., a copolymer where the probability of monomer X occupying a particular position of the copolymer increases or decreases towards opposite ends of the copolymer), and the like. The number of species of constituent units that make up a heteropolymer varies and can be any number, e.g., in some cases the number of species may range from 2-20, e.g., from 2 to 10, from 2to 5, from 2 to 4, from 4 to 10, or from 3 to 7.

Heteropolymers or copolymers may be “linear”, i.e., heteropolymers or copolymers that consist of a single main chain or “branched”, i.e., heteropolymers or copolymers that consist of at least two chains, e.g., a single main chain and at least one side chain. The number of side chains that make up a branched copolymer varies and can be any number and, e.g., in some cases may range from 1-20, e.g., from 1 to 10, from 1 to 5, from 1 to 3, from 2 to 4, from 4 to 10, or from 3 to 7.

As used herein the term “branched copolymer” may refer to a copolymer that contains two different homopolymers, e.g., a main chain homopolymer of monomer X and at least one side chain homopolymer of monomer Y. The term may also refer to a copolymer that contains a main chain homopolymer and at least one side chain heteropolymer, e.g., a main chain homopolymer of monomer X and at least one side chain heteropolymer of monomers Y and Z. The term may also refer to a copolymer that contains a main chain heteropolymer and at least one side chain homopolymer, e.g., a main chain heteropolymer of monomers Y and Z and at least one side chain homopolymer of monomer X. In some instances a monomer species may be present in both the main chain polymer and the side chain polymer, e.g., a main chain homopolymer of monomer X and at least one side chain heteropolymer of monomers X and Y or a main chain heteropolymer of monomers X and Y and at least one side chain homopolymer of monomer X. As such, branched heteropolymers or copolymers of the present disclosure may be graft copolymers, i.e., branched copolymers in which the side chains are structurally distinct from the main chain.

As used herein the term “branched copolymers” also may refer to special branched copolymers or combinations of special branched copolymers or combinations of non-special branched copolymers and special branched copolymers. Non-limiting examples of special branched copolymers include star copolymers, brush copolymers, comb copolymers, diblock copolymers, triblock copolymers, junction block copolymers, terpolymers, and the like.

As used herein the term “copolymer” may also refer to “stereoblock copolymers” or copolymers where a special structure is formed from repeating monomers such that blocks are defined by the tacticity of each block. Stereoblock copolymers include those copolymers that contain blocks of diads (e.g., meso diads and racemo diads), triads (e.g., isotactic triads, syndiotactic triads, and heterotactic triads), tetrads, pentads, and the like. For example, in certain embodiments, stereoblock copolymers may be or may include “eutactic polymers”, i.e. polymers consisting of eutactic macromolecules where the substituents of the eutactic macromolecules are arranged in a sequence or pattern along the polymer backbone. Examples of eutactic polymers include, but are not limited to, isotactic polymers, syndiotactic polymers, and the like. For example, in certain embodiments, stereoblock copolymers may be or may include “isotactic polymers”, i.e., polymers consisting of meso diads and containing isotactic macromolecules where the substituents of the macromolecules are all located on the same side of the macromolecular backbone. In certain embodiments, stereoblock copolymers of the present disclosure may be or may include “syndiotactic” or “syntactic polymers”, i.e., polymers consisting of racemo diads and containing syndiotactic macromolecules where the substituents of the macromolecules alternate positions along the backbone chain.

As used herein the term “stereoblock copolymers” may also refer to or may also include “atactic polymers”, i.e., polymers consisting of between 1 and 99 number percent meso diads and containing atactic macromolecules where the substituents of the atactic macromolecules are distributed randomly along the backbone chain.

Definitions related to polymers, or the assembly of polymers, of the present disclosure are taken to be those definitions commonly known to one skilled in the art. Such definitions may be found, e.g., in Whelan T. (1994) Polymer technology dictionary. London: Chapman & Hall, the disclosure of which is herein incorporated, in its entirety, by reference

### DETAILED DESCRIPTION

Assay devices that include a poly(acid) membrane are provided. Aspects of the devices include a solid support and a poly(acid) membrane on a surface of the support, where the poly(acid) membrane includes an affinity element. In using the assay devices, a sample is contacted with the poly(acid) membrane and then a signal is obtained from the membrane. Also provided are kits that find use in practicing the methods described herein. The compositions and methods described herein find use in a variety of different applications, including analyte detection applications.

Before the present invention is described in greater detail, it is to be understood that this invention is not limited to particular embodiments described, as such may, of course, vary. The invention encompasses various alternatives, modifications, and equivalents, as will be appreciated by those of skill in the art. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is noted that, as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order which is logically possible.

Any publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

### Devices

Aspects of the invention include assay devices having a solid support and a poly(acid) membrane positioned on a surface of the solid support, wherein the poly(acid) membrane includes an affinity element. The poly(acid) membrane with affinity element may vary. In some instances, the poly(acid) membrane includes a poly(acid) component adsorbed to a surface of a porous membrane support. The poly(acid) component may have a variety of configurations on the surface of the porous membrane component. For example, the poly(acid) component may be arranged as a film, e.g., coating or layer (including layer by layer) configuration on the surface of the porous membrane. Alternatively, the poly(acid) component may be configured as a plurality of polymeric brushes on a surface of the porous membrane. The surface of the porous membrane may be any surface, including an upper surface, the surface of the pores of the membrane, etc., where in some instances all surfaces of the membrane may be stably associated with, e.g., adsorbed to, the poly(acid) component.

Configurations of poly(acid) components configured as films may vary. For example, in some instances poly(acid) films configured in a coating configuration may be configured in a homopolymer coating. Homopolymer coating configurations are those poly(acid) films that may be composed of homopolymers, i.e., polymers derived from a single species of constituent unit. Homopolymer coatings also include those poly(acid) films that may be composed of a single species of heteropolymer or copolymer, i.e., a homo-heteropolymer coating.

In certain embodiments, poly(acid) films configured in a layer-by-layer configuration may be configured in a heteropolymer coating or a heteropolymer layer-by-layer configuration. Heteropolymer layer-by-layer configurations are those poly(acid) films that may be composed of two or more different heteropolymers. Heteropolymer layer-by-layer configurations also include those poly(acid) films that may be composed of at least two different species of homopolymers, i.e., a hetero-homopolymer.

Configurations of poly(acid) components configured as a plurality of polymeric brushes, i.e. poly(acid) polymeric brushes, may vary. For example, poly(acid) polymeric brushes may be configured in a homopolymer brush structure or a heteropolymer or copolymer brush structure. Homopolymer brush structures are those poly(acid) polymeric brushes that may be composed of a homopolymer. Homopolymer brush structures also include those poly(acid) polymeric brushes that may be composed of a single species of heteropolymer or copolymer, i.e., a homo-heteropolymer brush structure. Heteropolymer brush structures also includes those poly(acid) polymeric brushes that may be composed of at least two different species of homopolymers, i.e., a hetero-homopolymer brush structure.

The poly(acid) components of interest may include poly(acid) films and/or poly(acid) brushes composed of any convenient homopolymer or copolymer. Homopolymer and copolymer configurations may vary. Synthesis of homopolymers and copolymers may be controlled to produce any desired sequence or pattern of polymer blocks in order to produce a particular homopolymer or copolymer for use in the poly(acid) component.

Desired sequence or pattern of polymer blocks, whether unit blocks, e.g., in copolymers, or structural blocks, e.g., stereoblock polymers, may be achieved by any convenient method of polymer synthesis or assembly as described in, e.g., Braun et al. (2013) Polymer Synthesis: Theory and Practice. 5^{th} ed. Springer, Ciferri A. (2005) Supramolecular Polymers, 2^{nd} ed. CRC Press: Boca Raton, Fla., the disclosures of which are herein incorporated by reference. For example, in certain embodiments, desired sequence or pattern of polymer blocks may be achieved by the joining of unit blocks or structural blocks in a head to tail configuration. In certain embodiments, a desired sequence or pattern of polymer blocks may be achieved by the joining of unit blocks or structural blocks in a head to head configuration. In certain embodiments, a desired sequence or pattern of polymer blocks may be achieved by the joining of unit blocks or structural blocks in a tail to tail configuration.

Poly(acid) films may include those poly(acid) films synthesized by any convenient method. Methods useful in the synthesis of poly(acid) films vary but may include methods of adsorption of one or more polyelectrolytes (i.e., a homopolymer or copolymer with charged groups) onto a solid substrate, e.g., through the attachment of a polyelectrolyte to a substrate by means of electrical charge differences between the polyelectrolyte and the substrate. Methods useful in the synthesis of poly(acid) films may also include the subsequent attachment of a second polyelectrolyte to a first polyelectrolyte by means of a difference in electrical charge between the first and second polyelectrolytes. In certain instances, the attachment of the second polyelectrolyte to the first polyelectrolyte takes place after the first polyelectrolyte has attached to the substrate. In some embodiments, poly(acid) films may be composed of a single polyelectrolyte. In certain embodiments, poly(acid) films may be composed of two or more different polyelectrolytes, including e.g., 3 or more, 4 or more, 5 or more, 6 or more, 7 or more, 8 or more, 9 or more, or 10 or more.

Polyelectrolytes that find use in poly(acid) films may vary widely. For example, in some instances, such polyelectrolytes may represent anionic polyelectrolytes or polyanions, i.e., polyelectrolytes having a more negative charge as compared to the substrate or adjacent polyelectrolyte to which it is attached. In some instances, such polyelectrolytes may represent cationic polyelectrolytes or polycations, i.e., polyelectrolytes having a more negative charge as compared to the substrate or adjacent polyelectrolyte to which it is attached. As the charge of a particular polyelectrolyte may be dependent on characteristics of the solution in which the polyelectrolyte is dissolved, e.g., pH, a particular polyelectrolyte may be present as a polyanion or a polycation in different solutions, e.g., in solutions of different pH. As such, in certain instances, a polyelectrolyte may also be defined as a weak polyelectrolyte, e.g., having a pKa or pKb in the range of 2 to 10, or a strong polyelectrolyte, e.g., having a pKa or pKb outside the range of 2 to 10.

Anionic polyelectrolytes that find use in poly(acid) films include, but are not limited to, those available from commercial suppliers. For example, in certain embodiments, anionic polyelectrolytes are those available from Sigma-Aldrich (St. Louis, Mo.), such as poly(2-acrylamido-2-methyl-1-propanesulfonic acid), poly(2-acrylamido-2-methyl-1-propanesulfonic acid-co-acrylonitrile), poly(acrylic acid), polyanetholesulfonic acid, poly(sodium 4-styrenesulfonate), poly(4-styrenesulfonic acid), poly(4-styrenesulfonic acid-co-maleic acid), poly(vinyl sulfate), poly(vinylsulfonic acid), 4-styrenesulfonic acid, poly-L-glutamic acid, salts thereof and the like.

Cationic polyelectrolytes that find use in poly(acid) films include, but are not limited to, those available from commercial suppliers. For example, in certain embodiments, cationic polyelectrolytes are those available from Sigma-Aldrich (St. Louis, Mo.), such as poly(allylamine hydrochloride), poly(diallyldimethylammonium chloride), diallyldimethylammonium, poly(acrylamide-co-diallyldimethylammonium chloride), poly(2-dimethylamino)ethyl methacrylate), polyethylenimine, poly-L-glutamic acid, 8-anilino-1-naphthalenesulfonic acid, salts thereof and the like.

In certain embodiments, poly(acid) films derived from an anionic polyelectrolyte, e.g., poly(acrylic acid) (PAA), are adsorbed onto a substrate, e.g., a porous support, at low pH, e.g., at pH between 2 to 5, e.g., from pH 3 to 5, e.g., pH 3, pH 4, or pH 4.7. In certain embodiments an anionic polyelectrolyte is adsorbed directly to a substrate, e.g., PAA may be adsorbed directly to a porous membrane support. In some embodiments, an anionic polyelectrolyte is absorbed indirectly to a substrate, e.g., by means of an adhesion layer, e.g., PAA may be adsorbed to an adhesion layer that is adsorbed to a porous membrane support. Any convenient agent that attaches to a substrate to facilitate the subsequent attachment of a polyanion or polycation may find use as an adhesion layer. In some instances, agents that find use in adhesion layers may be those agents that form multiple hydrophobic interactions with a porous membrane support. Adhesion layer agents may vary widely but in some cases may include poly(styrene sulfonate) (PSS).

In certain embodiments, layer-by-layer configurations of poly(acid) films may include those poly(acid) films where an adhesion layer containing one or more adhesion layer agents, e.g., PSS, is first layered on a porous support. In certain embodiments, layer-by-layer configurations of poly(acid) films may include those poly(acid) films where one or more anionic polyelectrolytes, e.g., PAA, are first layered on a porous support, e.g., without the use of an adhesion layer. In certain embodiments, after the layering of one or more anionic polyelectrolytes, one or more cationic polyelectrolytes, e.g., protonated poly(allyl amine) (PAH), polyethyleneimine (PEI), etc., are layered on the anionic polyelectrolyte. In certain embodiments, a combination of two more polyelectrolytes are layered on a porous support, e.g., a combination of PAH and PAA or a combination of PEI and PAA, with or without the use of an adhesion layer. Accordingly, poly(acid) films may be simple or may be complex. Simple poly(acid) films will vary but may include those poly(acid) films that include a small number of poly electrolyte layers, e.g., one layer, two layers, or three layers. Complex poly(acid) films will vary but may include those poly(acid) films that include more than a small number of polyelectrolyte layers, e.g., 3 or more layers, e.g., 4 or more layers, 5 or more layers, 6 or more layers, 7 or more layers, 10 or more layers, 15 or more layers, or 20 or more layers. Any desired number or combination of layers may be constructed in the resulting poly(acid) film.

Poly(acid) polymeric brushes may include those poly(acid) polymeric brushes synthesized by any convenient method. For example, methods useful in the synthesis of poly(acid) polymer brushes include, but are not limited to: plasma polymerization, heat-assisted or UV-assisted graft polymerization, nitroxide-mediated polymerization, reversible addition-fragmentation chain-transfer polymerization, atom-transfer radical polymerization (ATRP), surface-initiated ATRP, and the like. Any particular method may be utilized, or parts of methods may be combined or exchanged, in order to achieve desired reaction characteristics. Such desired reaction characteristics may vary. For example, in some embodiments, desired reaction characteristics include, but are not limited to, polymerization in aqueous solution (e.g., polymerization in a solution that is not an organic solvent), minimized in solution polymerization (i.e., a high preference for polymerization of substrate bound polymers over non-substrate bound polymers), controlled polymer growth rate, efficient polymer growth, and low polydispersities (i.e. a small range of polymer sizes).

In certain embodiments, the poly(acid) polymeric brushes may be those synthesized by surface initiated ATRP, where ATRP is initiated through the attachment of an initiator to a substrate. In certain embodiments the substrate to which the initiator is attached may be the porous membrane support. In other embodiments, the substrate to which the initiator is attached may be an intermediate substrate upon which ATRP is initiated before, during, or after the intermediate substrate is attached to the porous membrane support. For example, in certain embodiments, the initiator is attached to an intermediate substrate, e.g., a polymer primer, after the intermediate substrate is attached to the porous support.

Intermediate substrates useful in mediating attachment of an ATRP initiator to a porous support may vary widely. Such intermediate substrates are those substrates that attach simultaneously to a primary substrate, e.g., a porous support, and to a component of a polymer, e.g., an initiator or a monomer. In some instances, an intermediate substrate may be a polymer. In certain instances adhesion layer agents may find use as intermediate substrates, e.g., PSS may be used as an intermediate substrate.

Initiators may vary and may be any convenient initiator capable of initiating polymerization, e.g., radical polymerization, e.g., ATRP. Polymerization initiators of interest include, but are not limited to, those available from commercial suppliers, e.g., Sigma-Aldrich (St. Louis, Mo.). Initiators of radical polymerization include, but are not limited to, those radical polymerization initiators disclosed in Denisov et al. (2005) Free Radical Initiators. John Wiley & Sons: New Jersey, the disclosure of which is herein incorporated by reference. In certain embodiments, radical polymerization initiators may also include silane initiators, e.g., trichlorosilane.

Examples of ATRP initiators that may find use in constructing poly(acid) components include, but are not limited to: bis[2-(2′-bromoisobutyryloxy)ethyl]disulfide, bis[2-(2-bromoisobutyryloxy)undecyl]disulfide, 2-bromoisobutyric anhydride, α-bromoisobutyryl bromide, 2(2-bromoisobutyryloxy)ethyl acrylate, 2-(2-bromoisobutyryloxy)ethyl methacrylate, tert-butyl α-bromoisobutyrate, 3-butynyl 2-bromoisobutyrate, dipentaerythritol hexakis(2-bromoisobutyrate), dodecyl 2-bromoisobutyrate, ethyl α-bromoisobutyrate, ethylene bis(2-bromoisobutyrate), 2-hydroxyethyl 2-bromoisobutyrate, 1-(DL-1,2-isopropylideneglyceryl) 2-bromoisobutyrate, methyl α-bromoisobutyrate, octadecyl 2-bromoisobutyrate, pentaerythritol tetrakis(2-bromoisobutyrate), 1-(phthalimidomethyl) 2-bromoisobutyrate, poly(ethylene glycol) bis(2-bromoisobutyrate), poly(ethylene glycol) methyl ether 2-bromoisobutyrate, propargyl 2-bromoisobutyrate, 1,1,1-tris(2-bromoisobutyryloxymethyl)ethane 10-Undecenyl 2-bromoisobutyrate, and the like.

In certain embodiments an initiator is further bound to one or more units of a polymer, e.g., a unit block, a monomer, or a macromonomer, in order to form a macroinitiator. Methods of constructing macroinitiators vary and in some cases a polymer may be post-polymerization modified with an initiator, e.g., an ATRP initiator, or in other cases a polymer may be copolymerized with an initiator, e.g., an ATRP initiator. Any convenient unit of a polymer may find use as an incorporation site of an initiator in order to from a macroinitiator. Suitable initiators may be incorporated into a macroinitiator at any desired number percentage of a formed polymer where higher percentages of initiator incorporation result in higher rates of subsequent polymerization, e.g., higher polymer density, and lower percentages of initiator incorporation result in lower rates of subsequent polymerization, e.g., a lower polymer density. For example, in some instances initiators, e.g., ATRP initiators, may be present at anywhere from 1 to 50% in the macroinitiator, e.g., from 1 to 30%, from 10 to 40%, from 10 to 30%, from 1 to 20%, from 15 to 25%, or from 10 to 20%.

In certain instances, a macroinitiator may include an initiator bound to a cationic and anionic polymer, e.g., a cationic polyelectrolyte or anionic polyelectrolyte. For example, a macroinitiator may include an initiator, e.g., 2-(2-bromoisobutyryloxy)ethyl acrylate (BIEA), bound to a cationic polymer, e.g., 2-dimethylamino)ethyl methacrylate (DMAEMA). In some instances, a macroinitiator is further modified to improve reactivity, e.g., an macroinitiator may be further modified, e.g., alkylated with an alkylating agent, e.g., methylated with a methylating agent, in order to form a modified macroinitiator, e.g., poly(DMAEMA-co-BIEA) may be alkylated with methyl iodide to generate the modified macroinitiator poly(2-trimethylammonium iodide)ethyl methacrylate-co-BIEA) (TMAEMA-co-BIEA). In some instances, a macroinitiator or modified macroinitiator of a poly(acid) component is directly attached to the porous support. In other instances, a macroinitiator or modified macroinitiator is attached to a porous sport through the use of an intervening layer or substrate, e.g., an adhesion layer or an intermediate substrate.

Poly(acid) layers and brushes finding use in embodiments of the invention include, but are not limited to, those described in: Jain et al., “Protein Purification with Polymeric Affinity Membranes Containing Functionalized Poly(acid) Brushes,” Biomacromolecules (Apr. 12, 2010): 11:1019-1026; Anuraj et al., “An All Aqueous Route to Polymer Brush-Modified Membranes with Remarkable Permeabilities and Protein Capture Rates,” J. Memb. Sci. (Feb. 1, 2012) 389: 117-125; Bhattacharjee et al., “Formation of High-Capacity Protein-Adsorbing Membranes Through Simple Adsorption of Poly(acrylic acid)-Containing Films at Low pH,” Langmuir (May 1, 2012): 28: 6885-6892; Jain et al., “Completely Aqueous Procedure for the Growth of Polymer Brushes on Polymeric Substrates,” Langmuir (2007) 23:11360-11365; the disclosures of which are herein incorporated by reference. Also of interest are the poly(acid) membranes published in United States Published Application No. 20130244338; the disclosure of which is herein incorporated by reference.

In some instances the poly(acid) component, e.g., a poly(acid) film or poly(acid) brushes may be present on a porous membrane support. In some instances the porous membrane support component is attached to a solid support. The porosity of the porous membrane support may vary as desired. For example, in embodiments where membrane flexibility is desired a membrane with high porosity may be used or in embodiments where membrane rigidity is desired a membrane with low porosity may be used. The average pore size of the pores of the membrane may also vary as desired and may range from, e.g., from 0.5 to 20 μm in diameter, including e.g., from 1 to 10 μm, from 1 to 5 μm, from 1 to 3 μm, from 1 to 2 μm, from 2 to 5 μm, from 2 to 4 μm, from 3 to 5 μm, or from 4 to 5 μm. In some instances, average pore size of a membrane may be chosen based on the size of the poly(acid) component adhered to the membrane. For example, where a smaller poly(acid) component, e.g., a small poly(acid) film, is adhered to a membrane with a smaller average pore size, e.g., from 1 to 2 μm in diameter, including e.g., 1.2 μm, may be used. In other instances where a larger poly(acid) component, e.g., a large poly(acid) brush, is adhered a membrane with a larger average pore size, e.g., from 3 to 6 μm in diameter, including e.g., 5 μm, may be used. The use of a large poly(acid) component may or may not require the use of a membrane with large average pore size. For example, in some instances, a large poly(acid) component may be used in conjunction with a membrane of small average pore size. Likewise, in some instances, a small poly(acid) component may be used in conjunction with a membrane of large average pore size.

Average pore size refers to the arithmetic mean of the size of the pores of a membrane. Any convenient standard measurement of pore size, e.g., pore diameter or pore volume, may be used in calculating average pore size. In some instances, average pore size may also be determined by directly measuring the size of a representative sample or a representative number of pores and one need not measure every pore of a membrane in order to determine the average pore size of a membrane. In some instances, average pore size may be determined indirectly by measuring a functional characteristic of a subject membrane and estimating pore size based on measurements of the same functional characteristic measured in a reference membrane of known average pore size. These indirect methods must also consider, and in some cases measure, the pore distribution or pore density in order to accurately determine average pore size. Pore size and pore distribution may be measured by any convenient method including, but not limited to: the bubble point method, mercury porosimetry, thermoporometry, permporometry, the absorption method, methods based on liquid or gas transport, microscopic methods (e.g., light microscopy, scanning electron microscopy, transmission electron microscopy, atomic force microscopy, etc.). Such methods include, but are not limited to; those described and reviewed in Khulbe et al. (2008) Synthetic polymeric membranes: characterization by atomic force microscopy. Berlin: Springer, the disclosure of which is incorporated herein by reference.

The porous membrane support may be made up of a variety of materials, including but not limited to: polymeric materials, e.g., nylons, plastics, etc. In some instances the porous membrane support and the solid support may be made of the same material. In some instances the porous membrane support and the solid support may be made of different materials.

In certain embodiments polyamides may be used as the porous membrane support. Polyamides useful as membranes of the present disclosure may vary and may be either natural occurring or synthetic. In certain embodiments, the polyamide membrane is a nylon membrane. Nylon membranes may be either hydroxylated or non-hydroxylated. In certain instances, surface groups, e.g., surface amide groups, of non-hydroxylated membranes, e.g., non-hydroxylated nylon membranes, may be activated by conversion to active surface groups to form a hydroxyl-functionalized membrane, e.g., conversion of surface amide groups on non-hydroxylated nylon membranes to N-methylol polyamide (nylon-OH) surface groups. Any convenient material may be used in the porous membrane support, including such non-limiting examples as: sulfone containing polymers, e.g., polysulfone, polyethersulfone, and the like; fluoropolymers, e.g., polyvinylidene fluoride and the like; cellulose polymers; and the like. As described herein materials of the porous membrane support are not limited to those materials which are stable in organic solvents, e.g., materials that normally dissolve or disassociate in organic solvents may also be used in the porous membrane support through the use of aqueous assembly.

The poly(acid) membrane further includes an affinity element. The affinity element is an element or component that displays binding affinity for a category of molecules or a specific molecule, e.g., an analyte. Affinity elements of interest include those that are members of a specific binding pair, i.e., are binding pair members. A “binding pair member” is one of a first and a second moiety, wherein the first and the second moiety have a specific binding affinity for each other. Together the first and second moiety can be referred to as a “binding pair,” and each moiety (first and second) of the binding pair is therefore a binding pair member. Accordingly, a molecule may be said to include a binding pair member. A molecule may also be said to include two or more binding pair members, each of which can be members of different binding pairs. As mentioned above, in some instances the affinity of a first binding pair member to a second binding pair member of a give binding pair is characterized by a K_{D} (dissociation constant) of 10⁻⁵ M or less, e.g., 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. “Affinity” refers to the strength of binding, increased binding affinity being correlated with a lower Kd.

Suitable binding pairs include, but are not limited to, antigen/antibody pairs. Antigen/antibody pairs may include, for example, but are not limited to natural epitope/antibody pairs (e.g., insulin epitope/anti-insulin), laboratory generated antigen/antibody pairs (e.g., digoxigenin (DIG)/anti-DIG; dinitrophenyl (DNP)/anti-DNP; dansyl-X/anti-dansyl; Fluorescein/anti-fluorescein; lucifer yellow/anti-lucifer yellow; rhodamine/anti-rhodamine, etc), peptide or polypeptide antigen/antibody pairs (e.g., FLAG, histidine tag, hemagglutinin (HA) tag, c-myc tag, glutathione S transferase (GST) tag, protein A, Strep-tag, maltose binding protein (MBP), chitin-binding domain (CBD), S-tag, calmodulin binding protein (CBP), tandem affinity purification (TAP) tag, SF-TAP tag, VSV-G tag, herpes simplex virus (HSV) epitope tag, V5 epitope tag, 6× HN epitope, KT3 epitope (Martin et al., Science, 255:192-194 (1992)), tubulin epitope peptide (Skinner et al., J. Biol. Chem., 266:15163-15166 (1991)), the T7 gene 10 protein peptide tag (Lutz-Freyermuth et al., Proc. Natl. Acad. Sci. USA, 87:6393-6397 (1990)), etc. and the antibodies each thereto), and the like (Brizzard (2008) BioTechniques 44:693-695).

Suitable binding pairs also include, but are not limited to, pairs that are not antigen/antibody pairs, e.g., metal ion affinity peptide/metal ion (e.g., metal ion affinity peptides, e.g., histidine tag, that bind to metal ions such as Ni⁺², Co⁺², Fe⁺³, Al⁺³, Zn⁺², Cu⁺², and the like.), GST polypeptide/glutathione, Strep-Tactin, MBP/maltose (or amylose), CBD/chitin, Avitag/Avidin, CBP/calmodulin, TAP/calmodulin and/or IgG, SF-TAP/Strep-Tactin, biotin/avidin, biotin/streptavidin, biotin/neutravidin, and the like. A “metal ion affinity peptide” or “metal ion affinity tag” is a peptide that binds preferentially to a metal ion (e.g., Ni⁺², Co⁺², Fe⁺³, Al⁺³, Zn⁺², Cu⁺², and the like). A “histidine tag” or “histidine-rich affinity peptide” is a metal ion affinity peptide that is rich in histidines (e.g., 6× His tag, HAT tag, 6× HN tag, and the like). A histidine tag can also specifically bind to an anti-His antibody.

Affinity elements may be, in some cases defined as non-specific affinity elements, e.g., those affinity elements that bind a category of molecules, or, in some instances, may be defined as specific affinity elements, e.g., those affinity elements that bind a specific molecule.

In some instances, the affinity element is a non-specific affinity element, such as a metal ion chelating ligand complexed with a metal ion which, e.g., which binds to any suitably tagged molecule, e.g., a tagged protein, in a given sample. The metal ion chelating ligand complexed with a metal ion may vary with respect to the ligand and the metal ion. Examples of ligands of interest include, but are not limited to: iminodiacetic acid (IDA), nitriloacetic acid (NTA), caboxymethylated aspartic acid (CM-Asp), tris(2-aminoethyl) amine (TREN), and tris-carboxymethyl ethylene diamine (TED). These ligands offer a maximum of tri-(IDA), tetra-(NTA, CM-Asp), and penta-dentate (TED) complexes with the respective metal ion. A variety of different types of metal ions may be complexed to the ligands of the subject compounds. Metal ions of interest can be divided into different categories (e.g., hard, intermediate and soft) based on their preferential reactivity towards nucleophiles. Hard metal ions of interest include, but are not limited to: Fe³⁺, Ca²⁺ and Al³⁺ and like. Soft metal ions of interest include, but are not limited to: Cu⁺, Hg²⁺, Ag⁺, and the like. Intermediate metal ions of interest include, but are not limited to: Cu²⁺, Ni²⁺, Zn²⁺, Co²⁺ and the like. In certain embodiments, the metal ion that is chelated by the ligand is Co²⁺. In certain embodiments, the metal ion of interest that is chelated by the ligand is Fe³⁺. Additional metal ions of interest include, but are not limited to lanthanides, such as Eu³⁺, La³⁺, Tb³⁺, Yb³⁺, and the like. In certain embodiments, the affinity element includes aspartate groups and is referred to as an aspartate-based metal ion affinity element, where such compositions include a structure that is synthesized from an aspartic acid, e.g., L-aspartic acid. Aspartate-based metal ion affinity elements include aspartate-based ligand/metal ion complexes, e.g., tetradentate aspartate-based ligand/metal ion complexes, where the metal ion complexes have affinity for proteins, e.g., proteins tagged with a metal ion affinity peptide. In some instances, aspartate-based compositions of the present disclosure include structures having four ligands capable of interacting with, i.e., chelating, a metal ion, such that the metal ion is stably but reversibly associated with the ligand, depending upon the environmental conditions of the ligand.

In certain embodiments, non-specific affinity elements include tag-binding affinity elements that directly bind a molecular tag, e.g., a protein tag, e.g., an epitope tag, or a substrate tag, e.g., a chemical tag. The tag-binding affinity element may vary with respect to the tag. For example, in some instances, the tag may be a polypeptide epitope tag, e.g., a FLAG epitope, and the tag-binding affinity element may be a polypeptide, e.g., an antibody, that directly binds the polypeptide epitope tag, e.g., an anti-FLAG antibody. Antibodies that bind polypeptide epitope tags include but are not limited to: anti-FLAG antibodies, anti-His epitope tag antibodies, anti-HA tag antibodies, anti-Myc epitope tag antibodies, anti-GST tag antibodies, anti-GFP tag antibodies, anti-V5 epitope tag antibodies, anti-6× His tag antibodies, anti-6× HN tag antibodies, and the like. Such antibodies are available from commercial suppliers, e.g., from Clontech (Mountain View, Calif.), Thermo Scientific (Rockford, Ill.), and the like.

In other instances, the tag may be a chemical substrate that directly binds with a binding partner. The chemical substrate may be any convenient chemical substrate with one or more binding partners. For example, the chemical substrate may be biotin and thus the tag-binding affinity element may be any binding partner of biotin, e.g., avidin, streptavidin, an anti-biotin antibody, and the like. Further examples of tag-binding affinity elements that bind chemical substrates include, but are not limited to, anti-horseradish peroxidase antibodies, anti-digoxigenin antibodies, anti-alkaline phosphatase antibodies, anti-fluorescein isothiocyanate antibodies, anti-tetramethylrhodamine antibodies, and the like. Such tag-binding affinity elements are available from commercial suppliers, e.g., from Thermo Scientific (Rockford, Ill.), Life Technologies (Carlsbad, Calif.), Sigma-Aldrich (St. Louis, Mo.), and the like.

In certain embodiments, the affinity element may be a specific affinity element, e.g., a specific affinity element is an immobilized molecule that specifically binds to another molecule (e.g., an analyte of interest, a competitor, and the like). In some embodiments, the affinity between a specific affinity element and the molecule to which it specifically binds when they are specifically bound to each other in a binding complex is characterized by a K_{D} (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. “Affinity” refers to the strength of binding, increased binding affinity being correlated with a lower Kd.

A variety of different types of specific binding agents may be employed as a specific affinity element. A specific affinity element is therefore considered to include a binding pair member (defined below). Specific binding agents that can be used as a specific affinity element include antibody binding agents, proteins, peptides (e.g., glutathione, epitopes, etc.), receptor ligands, haptens, nucleic acids (e.g., DNA sequences, PNA sequences, siRNA sequences, or RNA sequences), carbohydrates (e.g., amylose, maltose, polysaccharides), lectins, and the like.

The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′₂ fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. Methods of developing and using specific affinity elements, e.g., antibody affinity elements, are well known in the art, see e.g., Harlow & Lane (1999) Using Antibodies: A laboratory manual. Cold Spring Harbor Press: Cold Spring Harbor, N.Y. and Shepherd & Dean (2000) Monoclonal antibodies—practical approach. Oxford University Press: Oxford, UK, the disclosures of which are herein incorporated by reference. Furthermore, examples of assay devices utilizing specific affinity elements, which elements may find use in various embodiments of the present invention, include but are not limited to those disclosed in U.S. Patent Publication Nos. 20140093865 A1, 20140045727 A1, 20130203073 A1, 20130137598, 20120040336 A1 and 20100068826 and U.S. Pat. Nos. 4,632,901, 4,366,241 and 4,956,275, the disclosures of which are incorporated by reference herein.

In some embodiments, the poly(acid) membrane of the assay device includes multiple different affinity elements, each of which specifically binds to a different binding pair member. As a non-limiting example, a poly(acid) membrane can contain all or any combination of the following: anti-histidine tag antibody, immobilized metal ions (e.g., Ni⁺², Co⁺², Fe⁺³, Al⁺³, Zn⁺², Cu⁺²), glutathione, maltose, amylose, chitin, avidin, streptavidin, neutravidin, calmodulin, anti-V5 tag antibody, anti-c-myc tag antibody, anti-HA tag antibody, anti-HSV tag antibody, anti-TAP tag antibody, and the like.

A given device may include a single poly(acid) membrane functionalized with an affinity element, e.g., as described above, or it may include two or more such components such as three or more, four or more, five or more membranes, as desired, e.g., positioned on different areas of the solid support. In certain embodiments, multiple membranes may be separated by spacers. Where desired, the device may further include one or more additional poly(acid) membranes that lack an affinity element on the surface of the solid support, wherein such additional non-affinity element membranes may serve a variety of purposes, e.g., as a control, during use of the device.

Devices that make use of multiple poly(acid) membranes may vary and in some instances may include poly(acid) membranes with different attached affinity elements. Such different affinity elements may be attached to the same poly(acid) membrane or may be attached to different poly(acid) membranes. In some instances where different affinity elements are attached to a single poly(acid) membrane the different affinity elements may be integrated or mixed or evenly distributed such that binding of analytes to the different affinity elements occurs in overlapping areas of the device, e.g., completely overlapping area. In certain instances where different affinity elements may be attached to a single poly(acid) membrane the different affinity elements may be physically separated or spatially partitioned or separately grouped such that binding of analytes to the different affinity elements occurs in separate areas of the device, e.g., completely separate areas or non-overlapping areas. In some instances different affinity elements may be partially mixed and partially separated such that binding of analytes to the different affinity elements occurs in partially overlapping areas of the device.

In some instances the different affinity elements may bind the same analyte but with different affinity thus allowing qualitative or quantitative assessments to be made about the amount or concentration of the analyte present in the sample. For example, different affinity elements that bind the same analyte may differ in affinity for the analyte in as much as one affinity element binds the analyte with 1.1 to 100 times the affinity of the other affinity element, including e.g., 1.1 to 1.2 times, 1.2 to 1.3 times, 1.3 to 1.4 times, 1.4 to 1.5 times, 1.5 to 1.6 times, 1.7 to 1.8 times, 1.8 to 1.9 times, 1.1 to 1.5 times, 1.5 to 2 times, 2.5 to 3 times, 3 to 3.5 times, 3.5 to 4 times, 4 to 4.5 times, 4.5 to 5 times, 5 to 6 times, 6 to 7 times, 7 to 8 times, 8 to 9 times, 9 to 10 times, 2 to 4 times, 3 to 5 times, 2 to 5 times, 5 to 10 times, 10 to 15 times, 15 to 20 times, 10 to 20 times, 20 to 30 times, 30 to 40 times, 40 to 50 times, 50 to 60 times, 60 to 70 times, 70 to 80 times, 80 to 90 times, 90 to 100 times. In some instances different affinity elements that bind the same analyte may differ in affinity for the analyte as much as one affinity element binds the analyte with more than 100 times the affinity of the other affinity element.

In some instances the different affinity elements may bind different analytes thus allowing multiple assessments to be made from a single sample applied to a single assay device. The number of different affinity elements present on a single device may vary widely and in some cases may include from 2 to1000 different affinity elements, including e.g., from 2 to 5, from 3 to 6, from 4 to 7 from 5 to 8, from 5 to 10, from 10 to 20, from 20 to 30, from 30 to 40, from 50 to 60, from 60 to 70, from 80 to 90, from 90 to 100, from 100 to 200, from 200 to 300, from 300 to 400, from400 to500, from 500 to 600, from 600, to 700, from 700 to 800, from 800 to 900, and from 900 to 1000. In some instances the number of affinity elements present on a single device may be more than 1000, including e.g., more than 10,000, or more than 100,000.

According to certain embodiments, the poly(acid) membranes with attached different affinity elements or the different affinity elements themselves may be arrayed on a solid support such that they are physically addressable, e.g., the membranes or affinity elements may be arranged side-by-side or in a grid pattern, as such arrangements allow for the rapid assessment of detection of multiple analytes by simply determining which poly(acid) membrane or affinity element or which physically addressable space thereof has produced a detectable signal. Such assessments may be made simply by observing the device or by subjecting the device to a detector or reader as such methods are described herein.

In some instances the concentration or amount of affinity element attached to the poly(acid) membrane is known and thus may be correlated with a known standard to allow for a quantitative assessment of the amount of particular analyte or multiple analytes present in a sample. For example, in some instances multiple locations on an assay device may contain multiple different known concentrations of the same affinity element such that depending on which particular location or locations of the assay device generate a detectable signal a quantitative assessment of the concentration of the analyte in the sample may be made. In making the quantitative assessment a signal or multiple signals generated on or from the assay device may be compared to a reference standard. Reference standards for quantitate assessments may vary and in some cases may be provided with an assay device, e.g., included in the packaging of an assay device, e.g., in printed form or computer readable form, or included directly on the assay device, e.g., printed on a surface of the assay device, e.g., a front surface or a back surface.

Detection of a bound analyte may be achieved through the use of a signal detection system. Signal detection systems of the present disclosure may vary and in some instances may include a reporter binding member. Reporter binding members of the present disclosure may vary and in some instances may include moieties that directly bind the analyte. For example, a reporter binding member may comprise a moiety that binds to an analyte, e.g., a peptide or a protein, at a site on the analyte that is different from the site where the affinity element binds the analyte, e.g., the affinity element and the reporter binding element may bind different tags. Such binding, in some instances, may be such that the reporter binding member and the affinity element “sandwich” the analyte. In some instances, the reporter binding member and the affinity element may bind to the same moiety of the analyte but at separate sites on the moiety, e.g., separate sites on the same tag.

In some instances the reporter binding member may directly bind the affinity element essentially only when the affinity element is also bound to an analyte. For example, a reporter binding member may utilized that has low binding affinity for an unbound affinity element but high binding affinity for a bound affinity element. In certain instances a reporter binding member may directly bind the analyte essentially only when the analyte is also bound to an affinity element. For example, a reporter binding member may utilized that has low binding affinity for an unbound analyte but high binding affinity for a bound analyte.

In some instances the reporter binding member directly binds the analyte with nearly equal affinity whether or not the analyte is bound to an affinity element or the analyte itself may serve as the reporter binding member. In such instances the high binding properties of the poly(acid) membrane facilitate the generation of a sufficiently high local concentration of reporter binding member or analyte bound to the poly(acid) membrane to allow for detection.

In some instances the reporter binding member is bound to the analyte prior to the analyte binding the affinity element, e.g., pre-bound. In certain cases the analyte may be generated or produced pre-bound to the reporter binding member, e.g., a protein tag generated during protein synthesis may serve as a reporter binding member. In other cases, a reporter binding member may be pre-bound to an analyte in solution, e.g., a reporter binding member may be coupled, e.g., chemically coupled, to an analyte.

The reporter binding member may further include a member of a signal producing system. The member of the signal producing system may vary widely depending on the particular nature of the assay device and may be any directly or indirectly detectable label. Suitable detectable labels for use in the devices and methods disclosed herein include any moiety that is detectable by spectroscopic, photochemical, biochemical, immunochemical, electrical, optical, chemical, or other means. For example, suitable labels include biotin for staining with labeled streptavidin conjugate, fluorescent dyes (e.g., fluorescein, Texas red, rhodamine, green fluorescent protein, and the like), radiolabels (e.g., ³H, ²⁵I, ³⁵S, ¹⁴C, or ³²P), enzymes (e.g., horseradish peroxidase, alkaline phosphatase and others commonly used in an ELISA), and colorimetric labels such as colloidal gold or colored glass or plastic (e.g., polystyrene, polypropylene, latex beads). Patents that describe the use of such labels include U.S. Pat. Nos. 3,817,837; 3,850,752; 3,939,350; 3,996,345; 4,277,437; 4,275,149; 4,313,734; 4,366,241; 4,373,932; 4,703,017; 4,770,853;. See also Handbook of Fluorescent Probes and Research Chemicals (6th Ed., Molecular Probes, Inc., Eugene, Oreg.). Radiolabels can be detected using photographic film or scintillation counters, fluorescent markers can be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

In addition, the assay device further includes a solid support. Solid supports of the present disclosure may vary and may be fabricated from any convenient material or combination of convenient materials, including but not limited to: synthetic or engineered materials, e.g., polymers (e.g., plastics, fibers, etc.), glass, metals, metal alloys, composites, etc., or natural materials and materials directly derived from natural materials, e.g., paper, biological materials (cells, tissue, bone, skin, hair, shell, etc.), stone, mineral, etc.

In certain embodiments the solid support is made of, or primarily from, one or more polymeric materials, including but not limited to polymeric materials, e.g., plastics, where the material may be opaque or transparent, as desired. Polymeric materials useful in fabricating the solid support include, but are not limited to, those polymeric materials, e.g., plastics, resins, etc., that are commonly used in research, industrial, and consumer product settings, including but not limited to: acetal, cyclic olefin copolymer, ethylene propylene diene monomer rubber, ethylene propylene rubber, ethylene-chlorotrifluoroethylene copolymer (Halar®), ethylene-tetrafluoroethylene (Tefzel), fluorinated ethylene propylene (Teflon®), fluorinated polyethylene, high impact polystyrene, high-density polyethylene, low-density polyethylene, modified polyphenylene ether, Permanox, polycarbonate, polyetherimide, polyethylene teraphthalate, polyethylene terephthalate, polyethylene terephthalate copolymer, polyfluoroalkoxy (Teflon®), polymethyl methacrylate (acrylic), polymethylpentene, polypropylene, polypropylene copolymer, polystyrene, polysulfone, polyvinylidenedifluoride, polyvinyl chloride, ResMer™, styrene acrylonitrile, tetrafluoroethylene, tetrafluoroethylene (Teflon®), Thermanox, thermoplastic elastomer, thermoplastic polyester polyurethane, Tritan™, and the like.

In some instances the solid support may be a rigid solid support, e.g., rigid polymers (e.g., rigid plastics), glass, rigid fibrous material, stone, rigid metals, rigid metal alloys, rigid composite materials, etc. In some instances the solid support may be a flexible or pliable solid support, e.g., pliable polymers (pliable plastics, pliable films, etc.), pliable fabrics, pliable tapes, pliable metals, pliable metal alloys, pliable composite materials, paper, pliable minerals, etc.

In some instances the solids support of the present disclosure may be coated. Coatings and substances used in coatings of the solid support may vary and, in some cases, may be a coating that improves or alter some property of the solid support in a desired manner to improve the function, e.g., an analyte detection function, of an assay device. For example, solid support coatings may improve the physical interaction of the assay device with particular liquids in a desired way, e.g., increase or decrease hydrophobicity of the solid support, increase or decrease hydrophilicity of the solid support, and the like. Alternatively, solid support coatings may alter other physical properties of the solid support, e.g., increase or decrease static charge of the solid support, increase or decrease the surface area of the solid support, increase or decrease insulative properties of the solid support, and the like.

In some instances coatings of the solid support may interact directly with the poly(acid) membrane component, e.g., to facilitate attachment of the poly(acid) membrane component. In some instances coatings of the solid support may interact indirectly with the poly(acid) membrane, e.g., to facilitate attachment of a component to which the poly(acid) component attaches or is grown from. In some instances a coating of the solid support may include an intermediate support.

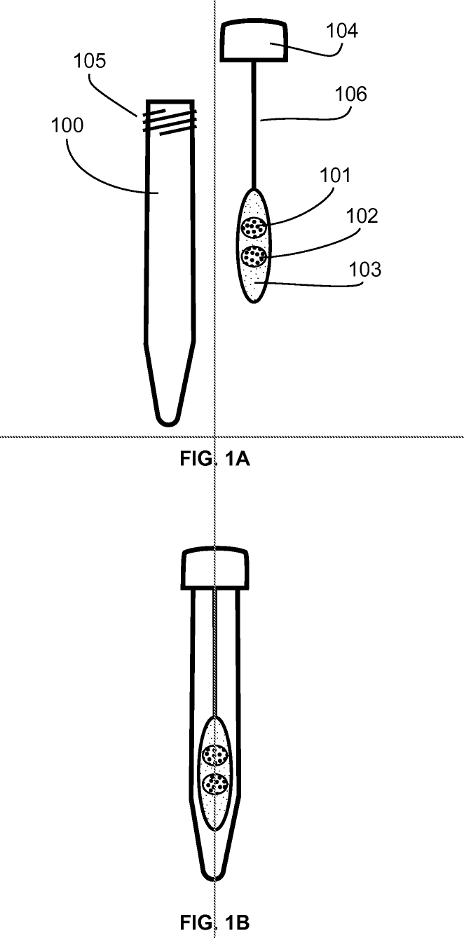

The solid support may have a variety of configurations. The support may or may not include through holes for allowing passage of fluid through the membrane element. Shapes of the solid support include, but are not limited to rectangular, square, circular, curvilinear, etc. in one embodiment of interest, the solid support is an elongated structure, e.g., a dipstick, such as illustrated in FIG. 1A-B.

In configurations shown in FIG. 1A, the elongated structure (103) is a dipstick configuration configured to be inserted into a tube (100), e.g., a standard laboratory vial or tube, e.g., a conical tube, a centrifuge tube, a culture tube, etc., as such tubes and vials are described herein. According to one embodiment the tube is a 15 mL conical tube having dimensions 17 mm in diameter by 120 mm in length and having screw threading (105) at the open end. The width and length of the elongated structure (103) may vary and may be dependent on the number and size of poly(acid) membranes (101 and 102) desired to be attached to the elongated support. For example, in some instances the length of the elongated structure may range from 0.5 cm to 12 cm in length, including, e.g., 0.5 cm to 5 cm, 5 cm to 10 cm, 0.5 cm to 2 cm, 1 cm to 3 cm, 2 cm to 4 cm, 3 cm to 5 cm, 4 cm to 6 cm, 5 cm to 7 cm, 6 cm to 8 cm, 7 cm to 9 cm, 8 cm to 10 cm, 9 cm to 11 cm, and 10 cm to 12 cm. In addition, in some instances, the width of the elongated structure may range from 0.1 cm to 1.7 cm, including, e.g., 0.1 cm to 0.9 cm, 0.8 cm to 1.7 cm, 0.1 cm to 0.3 cm, 0.2 cm to 0.4 cm, 0.3 cm to 0.5 cm, 0.4 cm to 0.6 cm, 0.5 cm to 0.7 cm, 0.6 cm to 0.8 cm, 0.7 cm to 0.9, 0.8 cm to 1 cm, 0.9 cm to 1.1 cm, 1 cm to 1.2 cm, 1.1 cm to 1.3 cm, 1.2 cm to 1.4 cm, 1.3 to 1.5 cm, 1.4 cm to 1.6 cm, and 1.5 cm to 1.7 cm.

In certain instances, the elongated structure may have attached only one poly(acid) membrane or may have attached more than one poly(acid) membrane, e.g., ranging from 2 to 20 poly(acid) membranes, including e.g., 2 to 10 membranes, 10 to 20 membranes, 2 to 5 membranes, 5 to 10 membranes, 10 to 15 membranes, 15 to 20 membranes, 2 membranes, 3 membranes, 4 membranes, 5 membranes, 6 membranes, 7 membranes, 8 membranes, 9 membranes, 10 membranes, 11 membranes, 12 membranes, 13 membranes, 14 membranes, 15 membranes, 16 membranes, 17 membranes, 18 membranes, 19 membranes, and 20 membranes. Where multiple membranes are present on a single elongated structure, the membranes may be the same or different. In certain instances multiple membranes differ in that one membrane is a test membrane (101) positioned adjacent to a control membrane (102). For example, a test membrane may be a fully functional membrane of an assay device and a control membrane may lack one or more components, e.g., an affinity element, in comparison to the test membrane such that the control membrane is a non-functional membrane in terms of detection of the analyte of the test membrane. In certain instances, the elongated structure may further include markings, e.g., markings indicating the identity of a single membrane or the identities of multiple membranes, such as text indicating the “test” membrane or the “control” membrane or symbols, such as “+” and/or “−” which correspond to symbols provided on corresponding instructions that may be provided in accordance with embodiments described herein.

As further depicted in FIG. 1A, in certain embodiments, the dipstick configuration may or may not include a stick (106) which joins the elongated structure and, if present, the cap (104). In certain instances, e.g., in the absence of a cap, the stick may also serve as a handle. In some embodiments, the stick serves to position the elongated structure at a desired position within a tube or vial when the dipstick is placed within the tube or vial, e.g., as depicted in FIG. 1B. Accordingly, the length of the stick may vary depending on the desired position of the elongated structure within the tube or vial, such that, in some embodiments, the length of the stick may range from 0.5 cm to 12 cm in length, including, e.g., 0.5 cm to 5 cm, 5 cm to 10 cm, 0.5 cm to 2 cm, 1 cm to 3 cm, 2 cm to 4 cm, 3 cm to 5 cm, 4 cm to 6 cm, 5 cm to 7 cm, 6 cm to 8 cm, 7 cm to 9 cm, 8 cm to 10 cm, 9 cm to 11 cm, and 10 cm to 12 cm.

In certain instances, the dipstick configuration may include a cap at one end (104), where the cap is configured to mate with the tube or vial, e.g., the cap is a screw cap. Accordingly the cap may contain screw threading that is compatible with screw threading present on the tube or the vial. In certain embodiments, mating the cap with the tube or vial creates a liquid-tight seal such that the tube or vial containing the dipstick may be agitated, e.g., rocked, nutated, shook, etc., or inverted without spilling.

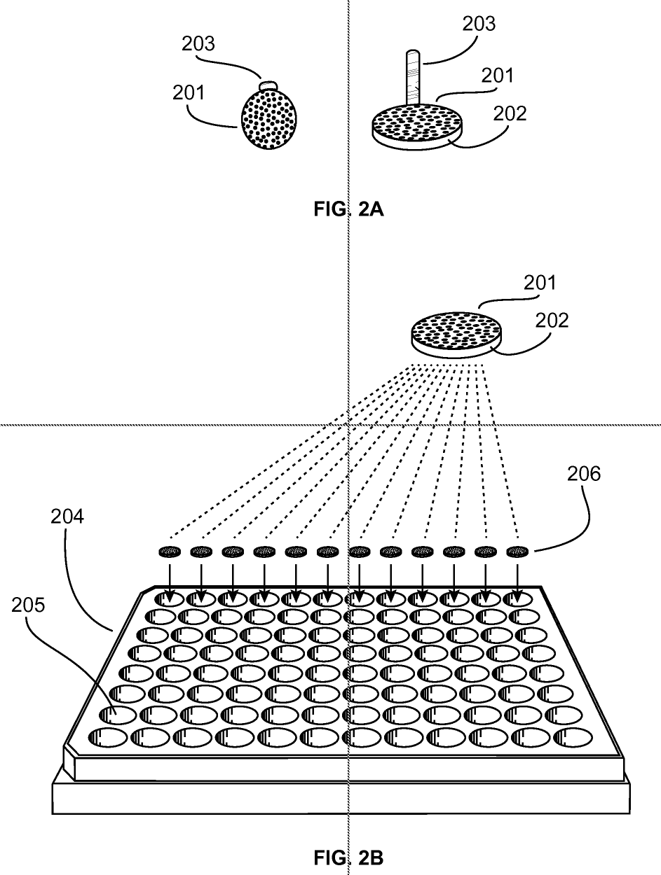

In another embodiment of interest, in configurations shown in FIG. 2A the solid support (202) is round, e.g., a disk, and may or may not contain additional attached structures, e.g., a handle (203) for holding or for dipping the device. In certain embodiments having a round solid support, the poly(acid) membrane (201) may also be round and may or may not be the same size, e.g., the same diameter, as the solid support. Round solid supports may vary greatly in size and may range from 1 mm to 1 m in diameter, including e.g., 1 mm to 1 cm, 1 cm to 10 cm, 10 cm to 100 cm, 100 cm to 200 cm, 200 cm to 300 cm, 300 cm to400 cm,400 cm to 500 cm,500 cm to 700 cm, and 700 cm to 1 m. In some instances, round configurations, as shown in FIG. 2B, including a poly(acid) membrane (201) and a solid support (202) that may be configured to be inserted into a well (205) of a multi-well plate (204). Multi-well plates may vary and in some instances may include, but are not limited to, 6 well plates, 12 well plates, 24 well plates, 48 well plates, 96 well plates, 384 well plates, and 1536 well plates. Accordingly, in some instances, a plurality of devices (206) may be either individually inserted into wells or arrayed for simultaneous insertion into wells of a multi-well plate. In certain instances, the solid support of assay devices configured for use with multi-well plates are not round, e.g., are some shape other than round, e.g., rectilinear.

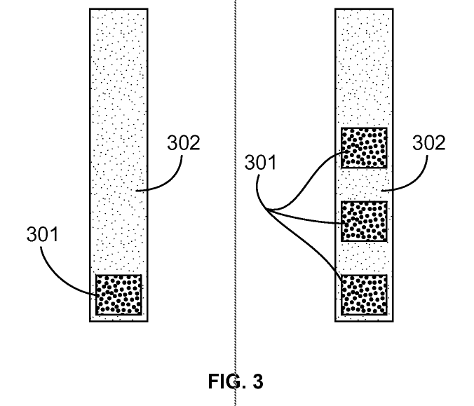

In some instances, in configurations shown in FIG. 3, the solid support (302) is a strip, e.g., a test strip or tape which can be cut into strips. Assay devices having a strip configuration may contain a single poly(acid) membrane (301). In certain instances, the strip may have attached more than one poly(acid) membrane (301), e.g., ranging from 2 to 100 poly(acid) membranes, including e.g., 2 to 10 membranes, 10 to 0 membranes, 2 to 5 membranes,5 to 10 membranes, 10 to 20 membranes, 0 to 40 membranes, 40 to 60 membranes, 60 to 80 membranes, 80 to 100 membranes, 2 membranes, 3 membranes, 4 membranes,5 membranes, 6 membranes, 7 membranes, 8 membranes, 9 membranes, 10 membranes, 11 membranes, 12 membranes, 13 membranes, 14 membranes, 15 membranes, 16 membranes, 17 membranes, 18 membranes, 19 membranes, and 20 membranes, 30 membranes, 40 membranes, 50 membranes, 60 membranes, 70 membranes, 80 membranes, 90 membranes, 100 membranes. Where multiple membranes are present on a single elongated structure, the membranes may be the same or different. In certain instances, a strip or tape configuration may further include perforations such that individual strips or tape pieces may be easily separated from a larger strip or longer tape, e.g., a role of tape. In certain instances, devices having a strip or tape configuration may be used in conjunction with a multi-well plate, e.g., as depicted for use with round solid supports in FIG. 2B.

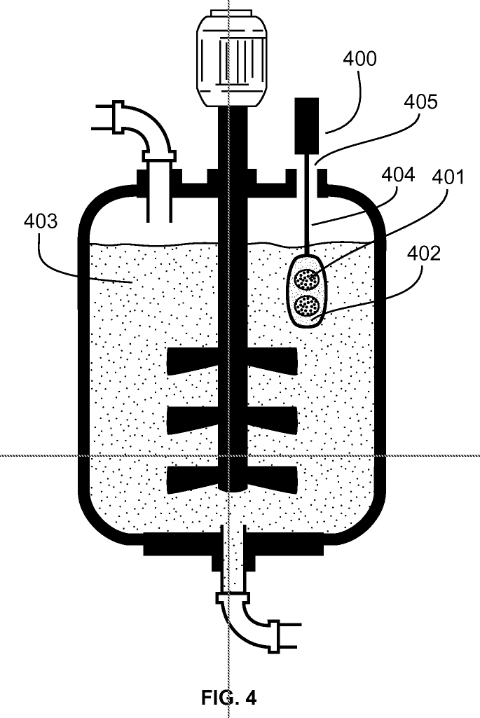

Assay devices of the present disclosure may be configured to be compatible with a wide range of vessels as described herein. For example, the solid support may be configured to be positioned into small vessels, e.g., similar to those depicted in FIG. 1A-B as well as large vessels, e.g., such as a fermenter or bioreactor as shown FIG. 4. In some instances, in configurations shown in FIG. 4, the dipstick configuration may be adapted for use in large scale vessels containing large volumes of liquid (403) to be sampled. In some instances the components of a corresponding device include those components previously described for a dipstick configuration, including but not limited to, a handle (400), a stick (404), an elongated support (402), and one or more poly(acid) membranes (401). Such components may be present or absent as desired and according to the particular application. In certain embodiments, one or more of the listed components may be excluded. Configurations of assay devices according to FIG. 4 may, in some cases, be configured to be inserted through a sampling port (405) or other opening of a vessel and thus components of the device may be sized accordingly to fit into such an opening.

In certain instances, elongated structures of assay devices of the present disclosure may be configured to be compatible with a test tube or a microcentrifuge tube or a culture flask or a culture tube or a culture bottle.

In some instances the elongated structure may be configured to fit into a common laboratory tube, e.g., in a dipstick configuration. Common laboratory tubes include but are not limited to 0.5 mL microcentrifuge tubes, 1.5 mL microcentrifuge tubes, 2.0 mL microcentrifuge tubes,5 mL centrifuge/culture tubes, 13 mL centrifuge/culture tubes, 15 mL centrifuge/culture tubes, 50 mL centrifuge/culture tubes. Such conventional laboratory or industrial centrifuge tubes include those that are commercially available, e.g., from Eppendorf (Hamburg, Germany), BD Biosciences (San Jose, Calif.), Thermo Fisher Scientific (Rockford, Ill.), and the like. The actual dimensions of the elongated structure may vary depending on the specific dimensions of the intended tube and, e.g., in some instances the elongated structure may be configured for use in a 0.5 mL tube (e.g., 6.7 mm in diameter or less) or configured for use in a 1.5 mL tube (e.g., 9.8 mm in diameter or less) or configured for use in a 2.0 mL tube (e.g., 9.8 mm in diameter or less) or configured for use in a5 mL tube (e.g., 17 mm in diameter or less) or configured for use in a 15 mL tube (e.g., 17 mm in diameter or less) or configured for use in a 50 mL tube (e.g., 31 mm in diameter or less). In such configurations, the length of the elongated structure may vary an in some cases may be less than the overall height of the tube in order to allow the elongated structure to be secured, e.g., with a screw cap, inside the tube.

In certain embodiments the elongated structure is configured as a dipstick configured to be contacted with solution inside a collection bottle. Collection bottles may vary and may be either specifically designed to be compatible with the elongated structure or may be any conventional laboratory bottle that is compatible with the elongated structure. For example, conventional laboratory bottles, e.g., those laboratory bottles configured to be compatible with a conventional laboratory or industrial centrifuge, include, but are not limited to, 100 mL bottles, 175-25 mL conical bottles, 50 mL flat bottom bottles,400 mL bottles,500 mL bottles, 750 mL bottles, 1 L bottles, 1.5 L bottles, 2 L bottles, and the like. Such conventional laboratory or industrial centrifuge bottles include, but are not limited to, those commercially available, e.g., from Eppendorf (Hamburg, Germany), BD Biosciences (San Jose, Calif.), Thermo Fisher Scientific (Rockford, Ill.), and the like. In such configurations, the length of the elongated structure may vary an in some cases may be less than the overall height of the bottle in order to allow the elongated structure to be secured, e.g., with a screw cap or snap cap, inside the bottle.

In some instances the solid support may be flat glass or plastic, e.g., a microscope slide, or may be configured to be attached to flat glass or plastic, e.g., through the use of adhesives.

The dimensions of the solid support may vary widely and can be chosen based on a variety of factors. For example, where the solid support is configured as a strip, the solid support has a length that is longer than its width. While any practical configuration may be employed, in some instances the length is longer than the width by 1.5 fold or more, such as 2-fold or more, e.g., 10 fold or more, including 20-fold or more. In some instances, where the solid support is configured as a dipstick the length of the solid support ranges from 0.5 to 50 cm, such as 1.0 to 20 cm, e.g., 2.0 to 30 cm, while the width ranges 0.1 to 5.0 cm, such as 0.5 to 2.5 cm, e.g., 1 to 2 cm. The thickness of the solid support may also vary, ranging in some instances from 0.01 to 2 cm, such as 0.1 to 1.0 cm, e.g., 0.1 to 0.5 cm.

### Methods

Aspects of the invention further include methods of assaying a sample with devices such as described above. In the methods, a sample is assayed by contacting the sample with a device; and obtaining a signal from the poly(acid) membrane to assay the sample. In some instances, contacting includes embodiments where the sample is passed through the poly(acid) membrane. In some instances, the method includes washing unbound sample components from the poly(acid) membrane. In some instances, the method further includes exposing the sample contacted poly(acid) membrane to a signal producing system. For example, in some instances an assay device of the present disclosure may be contacted with a sample and then subsequently contacted with one or more solutions that include one or more further components of the signal producing system, e.g., a reporter binding member, a label, a substrate used in producing a detectable signal, and the like.

In certain instances, the method may further include charging the poly(acid) membrane before use. As described herein, charging of a poly(acid) membrane describes contacting the poly(acid) membrane with a metal ion that may complex with a chelating ligand to form a metal ion affinity complex. Any convenient medium containing the desired metal ion with which the poly(acid) membrane is to be charged may be utilized in charging or recharging the poly(acid) membrane. For example, in certain instances salts, e.g., salts of chlorides or sulfates, of a desired metal ion, e.g., CuCl₂, NiCl₂, CuSO₄, or NiSO₄, are dissolved in water or buffer to generate a suitable medium for charging the poly(acid) membrane. Methods of contacting of the poly(acid) membrane with the charging medium may vary and in some instances may include incubating the poly(acid) membrane with the charging medium and/or flowing the charging medium through the poly(acid) membrane, e.g., by gravity, by vacuum pressure, by positive pressure. In certain instances, a poly(acid) membrane present in an assay device may have been previously charged with a particular metal ion, i.e., pre-charged, and subsequently stored before use in a ready-to-use format.

In some instances, the method may further include equilibrating the poly(acid) membrane prior to use. For example, an assay device may be contacted with one or more equilibration buffers. Equilibration buffers of the present disclosure may vary and are those buffers that prepare the poly(acid) membrane for the application of sample and optimal binding of the target to the affinity element. For example, in some instances, equilibration buffers of interest include but are not limited to solutions containing salts, e.g. sodium salts, e.g., sodium phosphate and/or sodium chloride, e.g., phosphate buffered saline (PBS). In some instances commonly used buffers may be employed, e.g., including but not limited to: Tris-HCl, Tris-acetate, HEPES, MOPS, sodium acetate, and the like. In some instances, chelating agents, e.g., ethylenediaminetetraacetic acid (EDTA), ethylene glycol tetraacetic acid (EGTA), citrate, etc., are excluded from, or if present are present in low amounts, equilibration buffers in order to increase binding of the target to the affinity element. In certain instances, an elution agent, e.g., imidazole, and/or a chelating agent is included in the equilibration buffer a low concentration, i.e., at a concentration lower than the concentration at which the agent would be used for elution of an analyte from the poly(acid) membrane, as a competitive binding agent in order to increase stringency of the poly(acid) membrane and decrease binding of undesired molecules, e.g., contaminates, to the affinity element.

Any convenient method of contacting the sample with the device may be employed. According to certain embodiments, the poly(acid) membrane may be completely or partially submerged in the sample. For example, in certain instances the sample to be assayed need not be partitioned from the sample containing substance in order to be assayed, e.g., an assay device that includes one or more poly(acid) membranes may be partially or completely submerged into the source of the sample. Accordingly, in one embodiment, where an environmental sample is to be assayed, contacting the sample with the device may be achieved by completely or partially submerging the ploy(acid) membrane(s) into a sample source, e.g., a body of water (e.g., a lake, a river, a pond, a stream, an ocean, a reservoir, a pool, a tank, an estuary, a bay, etc.). In another embodiment where a growth culture is to be assayed, e.g., a bacterial growth culture, a yeast growth culture, an algae growth culture, etc., contacting the sample with the device may be achieved by completely or partially submerging the ploy(acid) membrane(s) into the growth culture.

In certain methods, the sample may be first partitioned from the sample source prior to contacting the sample with the device. For example one or more samples may be collected, e.g., in an appropriate collection vessel or vessels, including but not limited to vials, tubes, jars, bottles, flasks, jugs, carboys, etc., prior to contacting the sample with the device. Such samples may be assayed, e.g., contacted with the assay device, immediately or may be stored prior to being assayed. Sample storage times may vary and will depend on, e.g., the stability of the analyte to be detected, the stability of the medium in which the analyte may or may not be present, storage conditions, etc. For example, in certain embodiments, storage times may range from 30 min. to 10 years, e.g., including 30 min. to 1 hour, 1 hour to 4 hours, 4 hours to 8 hours, 1 hour to 8 hours, 4 hours to 4 hours, 8 hours to 4 hours, 1 day to 2 days, 1 day to 1 week, 3 days to 1week, 1 week to 1 month, 1 month to 2 months, 2 months to 6 months, 2 months to a year, 6 months to a year, 1 year to 2 years, 1 year to 5 years, 2 years to5 years, 1year to 10 years,5 years to 10 years, and 8 years to 10 years.

In certain methods, samples are stored prior to being assayed in appropriate storage conditions. Such storage conditions may vary and in some cases may include but are not limited to: ambient storage conditions (e.g., room temperature, including, e.g., from 15° C. to 30° C., including, e.g., 16° C. to 28° C., 18° C. to 26° C., 19° C. to 24° C., and 20° C. to 22° C.); cold storage conditions (e.g., refrigerated conditions (e.g., ranging from 1° C. to 12° C., including, e.g., 1° C. to 10° C., 2° C. to 8° C., 3° C. to 6° C., and 4° C.), freezer conditions (e.g., ranging from −35° C. to 0° C., including, e.g., −30° C. to 0° C., −25° C. to 0° C., −20° C. to 0° C., −15° C. to 0° C., −10° C. to 0° C., −5° C. to 0° C., −15° C., and −20° C.), ultra-low freezer conditions (e.g., ranging from −165° C. to −40° C., including, e.g., −150° C. to −60° C., −130° C. to −50° C., −100to −70° C., −164° C., −150° C., −135° C. , −86° C., −80° C., and −60° C.), cryopreservation conditions (e.g., liquid phase nitrogen storage (e.g., ranging from −200° C. to −190° C., including, e.g., −196° C.) and vapor phase nitrogen storage (e.g., ranging from −190° C. to −135° C., including, e.g., −190° C. to −140° C., −185° C. to 140° C., −190° C. to −160° C., −165° C. to −135° C., −180° C. to −160° C., and −170° C. to −150° C.)); air-tight conditions; anhydrous conditions (e.g., desiccation conditions); hydrous conditions (e.g., humid conditions or wet conditions); oxygen free conditions (e.g., stored under nitrogen), light-protective conditions, etc.

In some instances, the method may further include dissolving or diluting a sample in binding buffer prior to applying the sample to an assay device of the present disclosure. In some instances the binding buffer may have the same components as the equilibration buffer and may, in some instances, have the same composition as the equilibration buffer. In some instances the binding buffer may differ from the equilibration buffer by the presence or absence of one or more components. In some instances the binding buffer may differ from the equilibration buffer in the amount of one or more components. For example, in some instances the binding buffer may include more or less elution agent than the equilibration buffer in order to modulate binding stringency as desired. In some instances, the binding buffer may include more or less of a particular additional agent present in any other buffer described herein in order to, e.g., increase or decrease a particular characteristic of the analyte in order to modulate binding stringency as desired.

In certain instances, contacting the sample with the device involves an appropriate contact time, e.g., an amount of time appropriate for the device to be exposed to a sufficient amount of a particular analyte that may or may not be present in a particular sample such that the amount of the particular analyte capable of binding the poly(acid) membrane is above the detection threshold of the device. Appropriate contact times of a sample with the device in order to achieve a desired detection, or a likely desired detection where the presence of a desired analyte is unknown, may be determined, e.g., determined empirically (i.e. an empirically determined contact time) or calculated hypothetically (i.e. an estimated contact time) by, e.g., a manufacturer of the device or a user of the device. Contact times of methods of the present disclosure will vary, e.g., depending on the concentration of the analyte in the sample, and in some cases may be essentially instant, e.g., less than 1 second, or may be non-instant, e.g., of a given period of time. Such non-instant contact times may vary widely and in some cases may range from 1 second to 1 year, including, e.g., from 1 to 5 sec., from 5 to 10 sec., from 10 to 20 sec., from 10 to 30 sec., from 10 to 60 sec., from 1 to 2 min., from 2 to 5 min., from 5 to 10 min., from 10 to 15 min., from 15 to 30 min., from 30 to 60min. from 30 to 90 min., from 1 to 1.5 hrs., from 1 to 2 hrs., from 1 to 5 hrs., from 2 to 4 hrs., from 4 to 8 hrs., from 8 to 12 hrs., from 12 to 4 hrs., from 12 to 48 hrs., from 1 day to 1 week, from 2 days to 2 weeks, from 1 to 2 weeks, from 2 weeks to 1 month, from 1 month to 2 months, from 1 month to 6 months, and from 6 months to 1 year.

In some instances contacting the sample with the device may further include incubating the sample in contact with the poly(acid) membrane under particular conditions or in the presence of particular regents in order to allow the analyte to bind the affinity agent. In certain instances such incubating may be performed either with or without diluting the sample or contacting the device with binding buffer. Such incubating may be performed after the sample is applied to the poly(acid) membrane by any convenient means as described herein, e.g., by dropping (e.g., pipetting), the sample onto the poly(acid) membrane or placing (e.g., dipping) the poly(acid) membrane into the sample and allowing the sample to come into full contact with the poly(acid) membrane. Such incubations may be performed at any convenient temperature to increase binding of the target or to decrease non-specific binding, e.g. at room-temperature (RT), at 4° C., between 0 and 4° C., between 4° C. and 10° C., between 10° C. and RT, between RT and 37° C., between 37° C. and 55° C., between 55° C. and 95° C., or above 95° C. Such incubations may be performed with or without agitation, e.g., stirring, rocking, nutating, shaking, rotating, etc.

As described briefly above, the sample may be applied to the device by any convenient means. Methods of applying a sample to the device may vary and in some instances may include but are not limited to: dripping the sample onto the device, flowing the sample onto or over the device, passing the sample through the device or a portion of the device, e.g., passing the sample through the poly(acid) membrane, pipetting a sample onto the device, etc. In certain instances where the sample is a biological sample the sample may applied directly from the subject, e.g., a human subject, and onto the device without first being collected into any container, e.g., where the sample is saliva the sample may be spat onto the device, where the sample is blood the sample may be bled onto the device, where the sample is urine the sample may be urinated onto the device, etc.

In certain instances where the device is used to assay the presence of an analyte produced by a cultured organism, contacting the device may be achieved by placing the device or a portion of the device, e.g., the poly(acid) membrane or a portion of the poly(acid) membrane, into the growth vessel or growth chamber of the cultured organism. For example, where a cultured organism, e.g., a bacterium, is grown in a chamber, e.g., a well of a multi-well plate, or a vessel, e.g., a culture tube, and used to produce a desired analyte, the device may be placed into the chamber or vessel with the organism, e.g., in order to indicate the presence of the desired analyte or to indicate the time at which the desired analyte is present in the growth medium, e.g., present above a particular threshold concentration. In certain instances, where a cultured organism is used to produce an analyte, the organism may be destroyed, e.g., lysed, in order to facilitate release of the analyte into the growth medium or any other desired buffer.

In certain instances, the method may further include one or more washes with one or more suitable wash solutions, e.g., water, buffers, media, etc. In certain instances the one or more suitable washes may be utilized to remove unbound sample or components of the unbound sample following contacting the sample with the device. Examples of unbound components that may be washed away by such washes include, but are not limited to: contaminates, undesired analytes, unbound reporter binding agents, unbound labels, unbound tags, debris, solutions (e.g., the sample solution in which the analyte of interest was present or binding buffer solutions or solutions having characteristics (e.g., pH, buffering capacity, lack of buffering capacity, salt concentration, etc.) that are incompatible with downstream methods, e.g., detection methods). According to certain embodiments the device may be contacted with the sample by dipping the device or a portion of the device into the sample and, following some amount of contact time, the device is removed and washed, e.g., rinsed or soaked with an appropriate wash buffer. According to certain embodiments the sample may be contacted with, e.g., applied to, the device and, following some amount of contact time, the device is washed, e.g., rinsed or soaked with an appropriate wash buffer. According to certain embodiments multiple washes may be performed and the number of washes may vary and may range from 2 to 10 washes, including, e.g., 2 washes, 3 washes, 4 washes, 5 washes, 6 washes, 7 washes, 8 washes, 9 washes, and 10 washes. In certain instances the multiple washes may vary in stringency, e.g., may be of increasing stringency, e.g., each successive wash is more stringent than the previous wash, or decreasing stringency, e.g., each successive wash is less stringent than the previous wash. Such washes, whether individual or multiple, may individually be performed with or without agitation, e.g., stirring, rocking, nutating, shaking, rotating, etc. In some instances, the stringency of a wash may be increased or decreased by changing the agitation conditions of the wash, e.g., the stringency of the wash or washes may be increased by increasing agitation or the stringency of the wash or washes may be decreased by decreasing agitation. In some instances multiple washes may be performed at multiple different temperatures, e.g., to vary the stringency of the washes, such that the difference in temperature between one or more washes may, e.g., range from 1° C. to 50° C., including, e.g., 1° C. to 2° C., 1° C. to 3° C., 1 to 4° C., 1° C. to 5° C., 2° C. to 5° C., 3° C. to 6° C., 5° C. to 10° C., 10° C. to 15° C., 15° C. to 20° C., 10° C. to 20° C., 20° C. to 30° C., 20° C. to 40° C., 30° C. to 50° C., and 10° C. to 50° C.

In some instances wash buffers may be the same as either the binding buffer and/or the equilibration buffer. In certain instances, a wash buffer will be different, either due to the presence or absence of a particular component or to the amount of a particular component, from the binding buffer or the equilibration buffer. In some instances the wash buffer may differ from the binding buffer or the equilibration buffer only in pH. In certain instances where multiple wash buffers are employed, the multiple wash buffers may differ in the presence or absence of one or more components, e.g., the presence or absence one or more additional agents described above, e.g., detergents, or the amounts of one or more components, e.g., wash buffers may contain differing amounts of an elution agent for increasing stringency. In certain instances, multiple wash buffers may differ only in pH.

In certain instances, the method may further include exposing the assay device to a signal producing system, as described elsewhere herein, in order to facilitate detection of a bound analyte. In certain instances the assay device, having been previously contacted with the sample, is contacted with one or more agents of a signal producing system. In embodiments where such agents of a signal producing system are present in solution, such contacting may, e.g., be performed by inserting, e.g., dipping or soaking, the assay device or a portion of the assay device into one or more solutions of a signal producing system. In embodiments where such agents of a signal producing system are present in solution, such contacting may, e.g., be performed by applying, e.g., dripping or pipetting or pouring, one or more solutions of the signal production system onto the assay device or a portion of the assay device. In some instances, exposing the assay device to the signal producing system may further include incubating, e.g., to allow a reporter binding member to bind, in a solution that is compatible with the binding of the reporter binding member to a component of the assay device. Such incubations may be performed at any convenient temperature to increase binding of the reporter binding member or to decrease non-specific binding, e.g. at room-temperature (RT), at 4° C., between 0 and 4° C., between 4° C. and 10° C., between 10° C. and RT, between RT and 37° C., between 37° C. and 55° C., between 55° C. and 95° C., or above 95° C. Such incubations may be performed with or without agitation, e.g., stirring, rocking, nutating, shaking, rotating, etc.

In certain instances, the method further includes contacting the assay device with one or more labels, e.g., directly detectable labels, indirectly detectable labels, or some combination of directly detectable and indirectly detectable labels, of the signal producing system. As described elsewhere herein, in some instances a label of the signal producing system may or may not be bound to the reporter binding member. In certain embodiments, components of the signal producing system, e.g., detectable labels, may be detected by any convenient means as described in greater detail below without further processing.

In certain instances, the method further includes performing a detection reaction in accordance with a particular signal producing system. In some instances, the method may further include pre-incubating the assay device, with bound indirectly detectable label, in one or more buffers to facilitate a subsequent detection reaction. Examples of such pre-incubations may vary and in some cases may include one or more pre-detection reaction buffers used to equilibrate the assay device to a particular final detection reaction buffer. Such pre-detection reaction buffers are useful in gradually adjusting reaction conditions, e.g., pH, viscosity, buffering capacity, etc., locally surrounding the poly(acid) membrane of the assay device. In certain instances pre-detection reaction buffers are not used and the device is instead contacted with the final detection reaction buffer without first contacting with a pre-detection reaction buffer.

In certain instances, the method further includes contacting the assay device or a portion of the assay device with a detection reaction buffer, also referred to herein as a final detection reaction buffer. Detection reaction buffers are those buffers that provide for the effective production of a detectable signal from a label, e.g., from an indirectly detectable label, in the presence of any other necessary detection reagents, e.g., substrates, e.g., enzyme substrates. Such buffers may vary and may depend on the type of detection reaction and the type of label to be detected. For example, in some instances the assay device may be contacted with detection reaction buffers including but not limited to: tyramide signal amplification buffer, alkaline phosphatase reaction buffer, horseradish peroxidase reaction buffer, and the like. In some instances the buffer may contain one or more necessary substrate for the detection reaction and in some instances one or more necessary substrates for the reaction are added separately.

According to particular embodiments, detection reactions may be performed under a variety of different reaction conditions. In some instances the rate of the detection reaction or the final signal to noise ratio of the detection reaction may be controlled by altering the detection reaction conditions, including e.g., the temperature of the reaction condition, the pH of the reaction condition, the viscosity of the reaction condition, the concentration of particular detection reaction components, e.g., reaction substrate concentrations, reaction enzyme concentrations, salt concentrations, metal concentrations, metal ion concentrations, other reaction agent concentrations, etc. The length of time for which the detection reaction proceeds may vary depending on the type of detection reaction and the particular reaction conditions. In some instances, the detection reaction is allowed to go to completion or to extinction; meaning all or essentially all of the reactable amount of a limiting agent of the reaction has been used. In some instances, the detection reaction may be stopped, e.g., after the signal of a test or a control detection reaction reaches some minimal or threshold detectable level or after some specified period of time. In certain instances where stopping the detection reaction is desired any convenient method of stopping the reaction may be utilized including, e.g., removing the assay device from the reaction buffer and/or washing the assay device with a wash buffer. In certain instances the detection reaction may be stopped by inhibiting the function of some component of the detection reaction, e.g., inhibiting the function of the enzyme, e.g., by altering the reaction conditions such that they are incompatible with the function of the enzyme. In certain instances, after the detection reaction the assay device is contacted with a suitable buffer, e.g., a wash buffer, a counterstain buffer, a fixation buffer, and the like, in order to prepare the assay device for further processing, e.g., for detection of the signal or measurement of the signal produced from the signal producing system.

In certain embodiments where a detection reaction is not necessary for detection of analyte binding, such assay devices may or may not be subjected to further process prior to detection or measurement of the analyte binding signal. In certain cases where assay devices have not been subjected to a detection reaction assay devices may be nonetheless further processed, e.g., by contacted with a suitable buffer, e.g., a wash buffer, a counterstain buffer, a fixation buffer, and the like, in order to prepare the assay device for further processing, e.g., for detection of the analyte binding signal or measurement of the analyte binding signal produced from analyte binding.

Detection of a signal produced from a signal producing system that indicates the presence of an analyte may be performed by any convenient means in accordance with the particular assay device. For example, in some instances detection may be performed simply by observing the device e.g., observing by eye under ambient light, observing by eye under a particular light required for observing a particular detectable signal, observing through an observation device, or by subjecting the device to a detector or reader.

In some instances where detection is performed by observing by eye the detectable signal may be that which is easily discernable, e.g., a color change, a change in opacity, a change in tint (e.g., a change from dark to light or a change from light to dark), and the like.

In some instances for detection performed by observing by eye, or with the aid of an observation device, under a particular light required for observing a particular detectable signal the type of light used will be constrained by the particular requirements of the signal to be detected. For example, a fluorescent signal produced by a fluorescent label may be observed under fluorescent light of a particular wavelength, e.g., 355 nm, 395 nm, 488 nm, 514 nm, 352 nm, 543 nm, 594 nm, 612 nm, 632 nm, 790 nm, etc., or within a particular range of wavelengths, e.g., from 300 to 350 nm, from 350 to 400 nm, from 400 to 450 nm, from 450 to 500 nm, from 500 to 550 nm, from 550 to 600 nm, from 600 to 650 nm, from 650 to 700 nm, from 700 to 750 nm, from 750 to 800 nm, from 300 to 400 nm, from 400 to 500 nm, from 500 to 600 nm, from 600 to 700 nm, from 700 to 800 nm, etc.

Observation devices that may be used in detecting a signal produced from a signal producing system include but are not limited to detection devices commonly used in research laboratories, e.g., high sensitivity cameras, microscopes, ultraviolet lights, etc. In certain instances the signal produced may require the use of such an observation device to facilitate detection. In certain instances the signal produced from a signal producing system may not be directly observed and may instead be detected through the use of a detector or scanner. In some instances although the signal is visible a detector or scanner may be used in order to quantify the signal, e.g., allowing quantitative analysis of analyte amounts or quantitative comparison of the binding of analytes, including multiple different analytes, to multiple poly(acid) membranes. Detectors and scanners that find use in the devices and methods of the present disclosure include but are not limited to, e.g., film based detectors, photospectrometers, laser scanners, photo scanners, document scanners, etc.

In some instances, the method further includes reusing and/or recharging the poly(acid) membrane. Methods of recharging the poly(acid) membrane may vary and in some cases may be essentially the same as method used in charging the membrane as disclosed herein. In some instances methods of recharging the poly(acid) membrane may be different from those described for charging the poly(acid) membrane, e.g., may require changes in solutions or particular components or component concentrations in order to compensate for reduced binding capacity of the poly(acid) membrane. In other instances the membrane may be directly reused without stripping/recharging, e.g., when the same target or analyte is to be bound. In some instances the solid support may be reused and the poly(acid) membrane replaced.

### Utility

The methods, devices, and kits of the invention find use in a variety of different applications and can be used to determine whether an analyte is present in a multitude of different sample types from a multitude of possible sources. Depending on the application and the desired output of the methods described herein, an analyte may be detected in a qualitative manner (“present” vs “absent”; “yes, above a predetermined threshold” vs “no, not above a predetermined threshold”; etc.) or a quantitative manner, as described above. Also as described above, many different types of analytes can be analytes of interest, including but not limited to: a tagged analyte, a nucleic acid analyte, a reporter protein, a viral vector, a lab contaminant, a sample contaminant, a toxin, an environmental contaminate, a food contaminate, an organism (e.g., a parasite), and the like. Further, samples can be from in vitro or in vivo sources, and samples can be non-diagnostic or diagnostic samples.

In practicing methods of the invention, the samples can be obtained from in vitro sources (e.g., extract from a laboratory grown cell culture) or from in vivo sources (e.g., a mammalian subject, a human subject, a research animal expressing a tagged analyte of interest, etc.). In some embodiments, the sample is obtained from an in vitro source. In vitro sources include, but are not limited to, prokaryotic (e.g., bacterial) cell cultures, eukaryotic (e.g., mammalian, fungal) cell cultures (e.g., cultures of established cell lines, cultures of known or purchased cell lines, cultures of immortalized cell lines, cultures of primary cells, cultures of laboratory yeast, etc.), tissue cultures, column chromatography eluants, cell lysates/extracts (e.g., protein-containing lysates/extracts, nucleic acid-containing lysates/extracts, etc.), viral cultures, and the like. In some embodiments, the sample is obtained from an in vivo source. In vivo sources include living multi-cellular organisms and can yield non-diagnostic or diagnostic samples.

In some embodiments, the analyte is a non-diagnostic analyte. A “non-diagnostic analyte” is an analyte from a sample that has not been obtained from or derived from a living multi-cellular organism, e.g., mammal, in order to make a diagnosis. In other words, the sample has not been obtained to determine the presence of one or more disease analytes in order to diagnose a disease or condition. Accordingly, in some instances, methods of the invention are non-diagnostic methods. “Non-diagnostic methods” are methods that do not diagnose a disease (e.g., sickness, diabetes, etc.) or condition (e.g., pregnancy) in a living organism, such as a mammal (e.g., a human). As such, non-diagnostic methods are not methods that are employed to determine the presence of one or more disease analytes in order to diagnose a disease or condition.

In certain embodiments, the methods are methods of determining whether a non-diagnostic analyte is present in a non-diagnostic sample. As such, the methods are methods of evaluating a sample in which the analyte of interest may or may not be present. In some cases, it is unknown whether the analyte is present in the sample prior to performing the assay. In other instances, prior to performing the assay, it is unknown whether the analyte is present in the sample in an amount that is greater than (exceeds) a predetermined threshold amount. In such cases, the methods are methods of evaluating a sample in which the analyte of interest may or may not be present in an amount that is greater than (exceeds) a predetermined threshold.

As a non-limiting example of a non-diagnostic use of an assay device of the present disclosure, an organism, e.g., bacterial cells or eukaryotic cells, may be engineered to express a protein of interest where the protein has also been engineered to be expressed with a tag, e.g., a His-tag, and the protein may be released into to the growth media, e.g., through lysis of the cells or through engineering the protein with an export signal, and an assay device, e.g., configured as a dipstick, may be contacted with the growth media to allow for the detection of the protein of interest or detection of a threshold amount of the protein of interest, e.g., to verify sufficient expression of the protein.

Aspects of the non-diagnostic methods include determining whether a non-diagnostic analyte is present in a non-diagnostic sample. Non-diagnostic samples can be obtained from in vitro sources, e.g., prokaryotic cell cultures (e.g., bacterial cell cultures); eukaryotic cell cultures (e.g., mammalian cell cultures); tissue cultures; non-diagnostic animal tissue samples or body fluids (i.e., such samples when not being used for diagnosis); column chromatography devices; and the like, or from in vivo sources (e.g., a sample obtained from living multicellular organism).

In some instances, non-diagnostic samples that are tested using assay device methods are samples generated in a research laboratory, for example, samples that are obtained from research experiments, including biotechnology research experiments (such as in vitro experiments that may or may not employ living cells, recombinant vectors, synthesized proteins, etc.). Examples of research experiment samples include, but are not limited to: cell and tissue cultures (and derivatives thereof, such as supernatants, lysates, and the like); non-diagnostic animal tissue samples and body fluids; non-cellular samples (e.g., column eluants; acellular biomolecules such as proteins, lipids, carbohydrates, nucleic acids, etc.; in vitro synthesis reaction mixtures; nucleic acid amplification reaction mixtures; in vitro biochemical or enzymatic reactions or assay solutions; or products of other in vitro and in vivo reactions; viral vector packaging supernatants; etc.). As used herein, research experiment samples exclude environmental samples, e.g., samples that are obtained from the environment in order to determine some quality or aspect of the environment, such as presence of one or more toxins, peptides, proteins, nucleic acids, or small molecules, and the like.

In some instances, non-diagnostic samples differ from a diagnostic sample by including components not found in diagnostic samples and/or lacking components found in diagnostic samples. In some instances, the contents of a non-diagnostic sample are readily determined because the non-diagnostic sample has been prepared from known starting materials in a research laboratory under defined and controlled conditions and protocols. In contrast, a physiological sample obtained for diagnostic purposes is inherently of unknown content, since individuals vary in terms genetic makeup and exposure to environment conditions.

In some embodiments, the analyte is a diagnostic analyte. A “diagnostic analyte” is an analyte from a sample that has been obtained from or derived from a living multi-cellular organism, e.g., mammal, in order to make a diagnosis. In other words, the sample has been obtained to determine the presence of one or more disease analytes in order to diagnose a disease or condition. Accordingly, the methods are diagnostic methods. As the methods are “diagnostic methods,” they are methods that diagnose (i.e., determine the presence or absence of) a disease (e.g., sickness, diabetes, etc.) or condition (e.g., pregnancy, infertility, immunity) in a living organism, such as a mammal (e.g., a human). As such, certain embodiments of the present disclosure are methods that are employed to determine whether a living subject has a given disease or condition (e.g., diabetes). “Diagnostic methods” also include methods that determine the severity or state of a given disease or condition.

Diagnostic analytes that find use in devices and methods of the present disclosure are those analytes useful in diagnosing a disease or disorder or condition of interest, including but not limited to: Acanthamoeba Infection, Acinetobacter Infection, Adenovirus Infection, ADHD (Attention Deficit/Hyperactivity Disorder), AIDS (Acquired Immune Deficiency Syndrome), ALS (Amyotrophic Lateral Sclerosis), Alzheimer's Disease, Amebiasis, Intestinal Entamoeba histolytica infection, Anaplasmosis, Anemia, Angiostrongylus Infection, Animal-Related Diseases, Anisakis Infection (Anisakiasis), Anthrax, Aortic Aneurysm, Aortic Dissection, Arenavirus Infection, Arthritis (e.g., Childhood Arthritis, Fibromyalgia, Gout, Lupus, (Systemic lupus erythematosus), Osteoarthritis, Rheumatoid Arthritis, etc.), Ascaris Infection (Ascariasis), Aspergillus Infection (Aspergillosis), Asthma, Attention Deficit/Hyperactivity Disorder, Autism, Avian Influenza, B virus Infection (Herpes B virus), B. cepacia infection (Burkholderia cepacia Infection), Babesiosis (Babesia Infection), Bacterial Meningitis, Bacterial Vaginosis (BV), Balamuthia infection (Balamuthia mandrillaris infection), Balamuthia mandrillaris infection, Balantidiasis, Balantidium Infection (Balantidiasis), Baylisascaris Infection, Bilharzia, Birth Defects, Black Lung (Coal Workers' Pneumoconioses), Blastocystis hominis Infection, Blastocystis Infection, Blastomycosis, Bleeding Disorders, Blood Disorders, Body Lice (Pediculus humanus corporis), Borrelia burgdorferi Infection, Botulism (Clostridium botulinim), Bovine Spongiform Encephalopathy (BSE), Brainerd Diarrhea, Breast Cancer, Bronchiolitis, Bronchitis, Brucella Infection (Brucellosis), Brucellosis, Burkholderia cepacia Infection (B. cepacia infection), Burkholderia mallei, Burkholderia pseudomallei Infection, Campylobacter Infection (Campylobacteriosis), Campylobacteriosis, Cancer (e.g., Colorectal (Colon) Cancer, Gynecologic Cancers, Lung Cancer, Prostate Cancer, Skin Cancer, etc.), Candida Infection (Candidiasis), Candidiasis, Canine Flu, Capillaria Infection (Capillariasis), Capillariasis, Carbapenem resistant Klebsiella pneumonia (CRKP), Cat Flea Tapeworm, Cercarial Dermatitis, Cerebral Palsy, Cervical Cancer, Chagas Disease (Trypanosoma cruzi Infection), Chickenpox (Varicella Disease), Chikungunya Fever (CHIKV), Childhood Arthritis, German Measles (Rubella Virus), Measles, Mumps, Rotavirus Infection, Chlamydia (Chlamydia trachomatis Disease), Chlamydia pneumoniae Infection, Chlamydia trachomatis Disease, Cholera (Vibrio cholerae Infection), Chronic Fatigue Syndrome (CFS), Chronic Obstructive Pulmonary Disease (COPD), Ciguatera Fish Poisoning, Ciguatoxin, Classic Creutzfeldt-Jakob Disease, Clonorchiasis, Clonorchis Infection (Clonorchiasis), Clostridium botulinim, Clostridium difficile Infection, Clostridium perfringens infection, Clostridium tetani Infection, Clotting Disorders, CMV (Cytomegalovirus Infection), Coal Workers' Pneumoconioses, Coccidioidomycosis, Colorectal (Colon) Cancer, Common Cold, Conjunctivitis, Cooleys Anemia, COPD (Chronic Obstructive Pulmonary Disease), Corynebacterium diphtheriae Infection, Coxiella burnetii Infection, Creutzfeldt-Jakob Disease, CRKP (Carbapenem resistant Klebsiella pneumonia), Crohn's Disease, Cryptococcosis, Cryptosporidiosis, Cryptosporidium Infection (Cryptosporidiosis), Cyclospora Infection (Cyclosporiasis), Cyclosporiasis, Cysticercosis, Cystoisospora Infection (Cystoisosporaiasis), Cystoisosporaiasis, Cytomegalovirus Infection (CMV), Dengue Fever (DF), Dengue Hemorrhagic Fever (DHF), Dermatophytes, Dermopathy, Diabetes, Diamond Blackfan Anemia (DBA), Dientamoeba fragilis Infection, Diphtheria (Corynebacterium diphtheriae Infection), Diphyllobothriasis, Diphyllobothrium Infection (Diphyllobothriasis), Dipylidium Infection, Dog Flea Tapeworm, Down Syndrome (Trisomy 21), Dracunculiasis, Dwarf Tapeworm (Hymenolepis Infection), E. coli Infection (Escherichia coli Infection), Ear Infection (Otitis Media), Eastern Equine Encephalitis (EEE), Ebola Hemorrhagic Fever, Echinococcosis, Ehrlichiosis, Elephantiasis , Encephalitis (Mosquito-Borne and Tick-Borne), Entamoeba histolytica infection, Enterobius vermicularis Infection, Enterovirus Infections (Non-Polio), Epidemic Typhus, Epilepsy, Epstein-Barr Virus Infection (EBV Infection), Escherichia coli Infection, Extensively Drug-Resistant TB (XDR TB), Fasciola Infection (Fascioliasis), Fasciolopsis Infection (Fasciolopsiasis), Fibromyalgia, Fifth Disease (Parvovirus B19 Infection), Flavorings-Related Lung Disease, Folliculitis, Food-Related Diseases, Clostridium perfringens infection, Fragile X Syndrome, Francisella tularensis Infection, Genital Candidiasis (Vulvovaginal Candidiasis (WC)), Genital Herpes (Herpes Simplex Virus Infection), Genital Warts, German Measles (Rubella Virus), Giardia Infection (Giardiasis), Glanders (Burkholderia mallei), Gnathostoma Infection, Gnathostomiasis (Gnathostoma Infection), Gonorrhea (Neisseria gonorrhoeae Infection), Gout, Granulomatous amebic encephalitis (GAE), Group A Strep Infection (GAS) (Group A Streptococcal Infection), Group B Strep Infection (GBS) (Group B Streptococcal Infection), Guinea Worm Disease (Dracunculiasis), Gynecologic Cancers (e.g., Cervical Cancer, Ovarian Cancer, Uterine Cancer, Vaginal and Vulvar Cancers, etc.), H1N1 Flu, Haemophilus influenzae Infection (Hib Infection), Hand, Foot, and Mouth Disease (HFMD), Hansen's Disease, Hantavirus Pulmonary Syndrome (HPS), Head Lice (Pediculus humanus capitis), Heart Disease (Cardiovascular Health), Heat Stress, Hemochromatosis, Hemophilia, Hendra Virus Infection, Herpes B virus, Herpes Simplex Virus Infection, Heterophyes Infection (Heterophyiasis), Hib Infection (Haemophilus influenzae Infection), High Blood Pressure, Histoplasma capsulatum Disease, Histoplasmosis (Histoplasma capsulatum Disease), Hot Tub Rash (Pseudomonas dermatitis Infection), HPV Infection (Human Papillomavirus Infection), Human Ehrlichiosis, Human Immunodeficiency Virus, Human Papillomavirus Infection (HPV Infection), Hymenolepis Infection, Hypertension, Hyperthermia, Hypothermia, Impetigo, Infectious Mononucleosis, Inflammatory Bowel Disease (IBD), Influenza, Avian Influenza, H1N1 Flu, Pandemic Flu, Seasonal Flu, Swine Influenza, Invasive Candidiasis, Iron Overload (Hemochromatosis), Isospora Infection (Isosporiasis), Japanese Encephalitis, Jaundice, K. pneumoniae (Klebsiella pneumoniae), Kala-Azar, Kawasaki Syndrome (KS), Kernicterus, Klebsiella pneumoniae (K. pneumoniae), La Crosse Encephalitis (LAC), La Crosse Encephalitis virus (LACV), Lassa Fever, Latex Allergies, Lead Poisoning, Legionnaires' Disease (Legionellosis), Leishmania Infection (Leishmaniasis), Leprosy, Leptospira Infection (Leptospirosis), Leptospirosis, Leukemia, Lice, Listeria Infection (Listeriosis), Listeriosis, Liver Disease and Hepatitis, Loa loa Infection, Lockjaw, Lou Gehrig's Disease, Lung Cancer, Lupus (SLE) (Systemic lupus erythematosus), Lyme Disease (Borrelia burgdorferi Infection), Lymphatic Filariasis, Lymphedema, Lymphocytic Choriomeningitis (LCMV), Lymphogranuloma venereum Infection (LGV), Malaria, Marburg Hemorrhagic Fever, Measles, Melioidosis (Burkholderia pseudomallei Infection), Meningitis (Meningococcal Disease), Meningococcal Disease, Methicillin Resistant Staphylococcus aureus (MRSA), Micronutrient Malnutrition, Microsporidia Infection, Molluscum Contagiosum, Monkey B virus, Monkeypox, Morgellons, Mosquito-Borne Diseases, Mucormycosis, Multidrug-Resistant TB (MDR TB), Mumps, Mycobacterium abscessus Infection, Mycobacterium avium Complex (MAC), Mycoplasma pneumoniae Infection, Myiasis, Naegleria Infection (Primary Amebic Meningoencephalitis (PAM)), Necrotizing Fasciitis, Neglected Tropical Diseases (NTD), Neisseria gonorrhoeae Infection, Neurocysticercosis, New Variant Creutzfeldt-Jakob Disease, Newborn Jaundice (Kernicterus), Nipah Virus Encephalitis, Nocardiosis, Non-Polio Enterovirus Infections, Nonpathogenic (Harmless) Intestinal Protozoa, Norovirus Infection, Norwalk-like Viruses (NLV), Novel H1N1 Flu, Onchocerciasis, Opisthorchis Infection, Oral Cancer, On Virus, Oropharyngeal Candidiasis (OPC), Osteoarthritis (OA), Osteoporosis, Otitis Media, Ovarian Cancer, Pandemic Flu, Paragonimiasis, Paragonimus Infection (Paragonimiasis), Parasitic Diseases, Parvovirus B19 Infection, Pediculus humanus capitis, Pediculus humanus corporis, Pelvic Inflammatory Disease (PID), Peripheral Arterial Disease (PAD), Pertussis, Phthiriasis, Pink Eye (Conjunctivitis), Pinworm Infection (Enterobius vermicularis Infection), Plague (Yersinia pestis Infection), Pneumocystis jirovecii Pneumonia, Pneumonia, Polio Infection (Poliomyelitis Infection), Pontiac Fever, Prion Diseases (Transmissible spongiform encephalopathies (TSEs)), Prostate Cancer, Pseudomonas dermatitis Infection, Psittacosis, Pubic Lice (Phthiriasis), Pulmonary Hypertension, Q Fever (Coxiella burnetii Infection), Rabies, Raccoon Roundworm Infection (Baylisascaris Infection), Rat-Bite Fever (RBF) (Streptobacillus moniliformis Infection), Recreational Water Illness (RWI), Relapsing Fever, Respiratory Syncytial Virus Infection (RSV), Rheumatoid Arthritis (RA), Rickettsia rickettsii Infection, Rift Valley Fever (RVF), Ringworm (Dermatophytes), Ringworm in Animals, River Blindness (Onchocerciasis), Rocky Mountain Spotted Fever (RMSF) (Rickettsia rickettsii Infection), Rotavirus Infection, RVF (Rift Valley Fever), RWI (Recreational Water Illness), Salmonella Infection (Salmonellosis), Scabies, Scarlet Fever, Schistosomiasis (Schistosoma Infection), Seasonal Flu, Severe Acute Respiratory Syndrome, Sexually Transmitted Diseases (STDs) (e.g., Bacterial Vaginosis (BV), Chlamydia, Genital Herpes, Gonorrhea, Human Papillomavirus Infection, Pelvic Inflammatory Disease, Syphilis, Trichomoniasis, HIV/AIDS, etc.), Shigella Infection (Shigellosis), Shingles (Varicella Zoster Virus (VZV)), Sickle Cell Disease, Single Gene Disorders, Sinus Infection (Sinusitus), Skin Cancer, Sleeping Sickness (African Trypanosomiasis), Smallpox (Variola Major and Variola Minor), Sore Mouth Infection (Orf Virus), Southern Tick-Associated Rash Illness (STARI), Spina Bifida (Myelomeningocele), Sporotrichosis, Spotted Fever Group Rickettsia (SFGR), St. Louis Encephalitis, Staphylococcus aureus Infection, Streptobacillus moniliformis Infection, Streptococcal Diseases, Streptococcus pneumoniae Infection, Stroke, Strongyloides Infection (Strongyloidiasis), Sudden Infant Death Syndrome (SIDS), Swimmer's Itch (Cercarial Dermatitis), Swine Influenza, Syphilis (Treponema pallidum Infection), Systemic lupus erythematosus, Tapeworm Infection (Taenia Infection), Testicular Cancer, Tetanus Disease (Clostridium tetani Infection), Thrush (Oropharyngeal Candidiasis (OPC)), Tick-borne Relapsing Fever, Tickborne Diseases (e.g., Anaplasmosis, Babesiosis, Ehrlichiosis, Lyme Disease, Tourette Syndrome (TS), Toxic Shock Syndrome (TSS), Toxocariasis (Toxocara Infection), Toxoplasmosis (Toxoplasma Infection), Trachoma Infection, Transmissible spongiform encephalopathies (TSEs), Traumatic Brain Injury (TBI), Trichinellosis (Trichinosis), Trichomoniasis (Trichomonas Infection), Tuberculosis (TB) (Mycobacterium tuberculosis Infection), Tularemia (Francisella tularensis Infection), Typhoid Fever (Salmonella typhi Infection), Uterine Cancer, Vaginal and Vulvar Cancers, Vancomycin-Intermediate/Resistant Staphylococcus aureus Infections (VISA/VRSA), Vancomycin-resistant Enterococci Infection (VRE), Variant Creutzfeldt-Jakob Disease (vCJD), Varicella-Zoster Virus Infection, Variola Major and Variola Minor, Vibrio cholerae Infection, Vibrio parahaemolyticus Infection, Vibrio vulnificus Infection, Viral Gastroenteritis, Viral Hemorrhagic Fevers (VHF), Viral Hepatitis, Viral Meningitis (Aseptic Meningitis), Von Willebrand Disease, Vulvovaginal Candidiasis (VVC), West Nile Virus Infection, Western Equine Encephalitis Infection, Whipworm Infection (Trichuriasis), Whitmore's Disease, Whooping Cough, Xenotropic Murine Leukemia Virus-related Virus Infection, Yellow Fever, Yersinia pestis Infection, Yersiniosis (Yersinia enterocolitica Infection), Zoonotic Hookworm, and Zygomycosis.

In certain embodiments, the methods are methods of determining whether an analyte is present in a diagnostic sample. As such, the methods are methods of evaluating a sample in which the analyte of interest may or may not be present. In some cases, it is unknown whether the analyte is present in the sample prior to performing the assay. In other instances, prior to performing the assay, it is unknown whether the analyte is present in the sample in an amount that is greater than (exceeds) a predetermined threshold amount. In such cases, the methods are methods of evaluating a sample in which the analyte of interest may or may not be present in an amount that is greater than (exceeds) a predetermined threshold.

Diagnostic samples include those obtained from in vivo sources (e.g., a mammalian subject, a human subject, and the like.) and can include samples obtained from tissues or cells of a subject (e.g., biopsies, tissue samples, whole blood, fractionated blood, hair, skin, and the like). In some cases, cells, fluids, or tissues derived from a subject are cultured, stored, or manipulated prior to evaluation and such a sample can be considered a diagnostic sample if the results are used to determine the presence, absence, state, or severity of a disease (see e.g., the diseases listed above) or condition (e.g., pregnancy, fertility, immunity, etc.) in a living organism.

In some instances, a diagnostic sample is a tissue sample (e.g., whole blood, fractionated blood, plasma, serum, saliva, and the like) or is obtained from a tissue sample (e.g., whole blood, fractionated blood, plasma, serum, saliva, skin, hair, and the like). An example of a diagnostic sample includes, but is not limited to cell and tissue cultures derived from a subject (and derivatives thereof, such as supernatants, lysates, and the like); tissue samples and body fluids; non-cellular samples (e.g., column eluants; acellular biomolecules such as proteins, lipids, carbohydrates, nucleic acids; synthesis reaction mixtures; nucleic acid amplification reaction mixtures; in vitro biochemical or enzymatic reactions or assay solutions; or products of other in vitro and in vivo reactions, etc.); etc.

The subject methods can be employed with samples from a variety of different types of subjects. In some embodiments, a sample is from a subject within the class mammalia, including e.g., the orders carnivore (e.g., dogs and cats), rodentia (e.g., mice, guinea pigs, and rats), lagomorpha (e.g. rabbits) and primates (e.g., humans, chimpanzees, and monkeys), and the like. In certain embodiments, the animals or hosts, i.e., subjects are humans.

In certain instances, assay devices and methods of the present disclosure may be used in the detection of analytes from environmental samples, i.e., samples derived from the environment. As used herein, environmental samples specifically exclude research samples or other samples derived in a laboratory setting for research purposes. Environmental samples from which an environmental analyte may be detected using the assay devices and methods described herein include but are not limited to air samples, particulate samples, water samples (i.e., rain water samples, freshwater samples, seawater samples), and soil samples. In certain instances, an environmental sample may be applied directly to the assay device for the detection of an environmental analyte as described herein without pre-processing of the sample. In some instances, and environmental sample is first processed, e.g., ground, diluted, concentrated, dissolved, adsorbed, etc., prior to being applied to an assay device.

In certain embodiments where the environmental sample is not a liquid sample the sample may be first dissolved or soaked in any convenient solvent compatible with the assay device as described herein. For example, an environmental soil sample may be first dissolved in water in order to facilitate application of the sample to the assay device. In some instances an environmental sample is prepared in the field by contacting a collection device from an article of interest. For example, a surface, e.g., a plant surface, a fruit surface, a vegetable surface, a building surface, a work surface, etc., may be contacted with a collection device, e.g., a swab (e.g., a cotton swab or a cloth swap), in order to prepare an environmental sample. Collection devices may vary and may be any convenient collection device. In some instances collection devices may contain liquid such that the sample is converted, e.g., dissolved, into a liquid sample upon collection. In some instances the collection device may allow for the transfer of an analyte into a liquid, e.g., a collection device may be soaked in a solvent, e.g., water or buffer or organic solvent, that is compatible with analytes and assay devices of the present disclosure.

### Kits

Aspects of the invention further include kits, where kits include one or more assay devices, e.g., as described above. In some embodiments, devices of the kits further include one or more assay components, such as buffers, vials, signal producing system reagents, etc. The various assay components of the kits may be present in separate containers, or some or all of them may be pre-combined into a reagent mixture.

Assay components of kits of the present disclosure may vary and may include one or more buffers including but not limited to: charging buffer (e.g., buffer containing an affinity element or a component of an affinity element useful in charging the poly(acid) membrane of the assay device as described herein), equilibration buffer (e.g., useful in equilibrating the poly(acid) membrane of the assay device as described herein), binding buffer (e.g., protein binding buffer useful in generating optimal condition for the protein binding to the affinity element), detection buffer (e.g., buffers useful in mediating detection of a bound reporter binding member or other detectable label e.g., staining buffer, substrate buffer, detection reaction buffer, labeling buffer, etc.), wash buffer (e.g., basic wash buffer, high stringency wash buffer, low stringency wash buffer, etc.), stain wash buffer (e.g., specific buffer for washing the assay device following a detection reaction).

Assay components may further include control devices and reagents. For example, in some instances one or more control assay devices are included. In some instances control reagents are also included. Control reagents may vary and in some cases may include but are not limited to: one or more reagents containing one or more known concentrations of one or more analytes of interest; one or more reagents containing one or more non-specific analytes, e.g., non-specific protein analytes; one or more reagents containing a substance known to bind any one of the binding members, e.g., a reagent known to bind the affinity element, a reagent known to bind the reporter binding member, etc.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An assay device, the device comprising:
a solid support; and
a poly(acid) membrane positioned on a surface of the solid support, wherein the poly(acid) membrane comprises an affinity element.

2. The device according to claim 1, wherein the poly(acid) membrane comprises a poly(acid) component adsorbed to a surface of a porous membrane support.

3. The device according to claim 2, wherein the poly(acid) component comprises a film.

4. The device according to claim 2, wherein the poly(acid) component comprises poly(acid) brushes.

5. The spin column according to claim 1, wherein the affinity element comprises a non-specific affinity element.

6. The device according to claim 5, wherein the non-specific affinity element comprises metal ion chelating ligand complexed with a metal ion.

7. The spin column according to claim 1, wherein the affinity element comprises a specific affinity element.

8. The device according to claim 7, wherein the specific affinity element comprises an antibody or binding fragment thereof.

9. The spin column according to claim 1, wherein the device further comprise a second poly(acid) membrane on a surface of the solid support, wherein the second poly(acid) membrane lacks an affinity element.

10. The device according to claim 1, wherein the solid support is an elongated structure.

11. The device according to claim 10 , wherein the elongated structure comprises a cap at a first end.

12. The device according to claim 11, wherein the cap is a screw cap.

13. The device according to claim 11, wherein the device is present in a vial having a first end configured to mate with the cap.

14. A method of assaying a sample, the method comprising:
contacting the sample with a device according to claim 1; and
obtaining a signal from the poly(acid) membrane to assay the sample.

15. The method according to claim 14, wherein the sample is passed through the poly(acid) membrane.

16. The method according to claim 14, wherein the method comprises washing unbound sample components from the poly(acid) membrane.

17. The method according to claim 14, wherein the method further comprises exposing the sample contacted poly(acid) membrane to a signal producing system.

18. The method according to claim 14, wherein the method is a method of assaying the sample for the presence of an analyte.

19. The method according to claim 18, wherein the analyte is a diagnostic analyte.

20. The method according to claim 18, wherein the analyte is a non-diagnostic analyte.

21. A kit comprising:
a device according to claim 1; and
a vial configured to house the solid support and a volume of a liquid sample.

22-23. (canceled)
